(19) United States
(12) Patent Application Publication
(10) Pub. No.: US 2008/0271210 A1
(43) Pub. Date: Oct. 30, 2008

(54) Oryza Sativa Lectin-Like Receptor Kinase 1 (Oslrk1), a Gene Involved in Plant Development

(75) Inventors: Tatiana Kolesnik, Singapore (SG); Ritu Bhalla, Singapore (SG); Rengasamy Ramamoorthy, Singapore (SG); Srinivasan Ramachandran, Singapore (SG)

Correspondence Address: ROTHWELL, FIGG, ERNST & MANBECK, P.C., 1425 K STREET, N.W., SUITE 800, WASHINGTON, DC, 20005, US

(73) Assignee: Temasek Life Sciences Laboratory National University of Singapore, Singapore (SG)

(21) Appl. No.: 11/664,458

(22) PCT Filed: Oct. 1, 2005
(86) PCT No.: PCT/SG05/00335

§ 371 Date: Feb. 26, 2008

Related U.S. Application Data
(60) Provisional application No. 60/614,549, filed on Oct. 1, 2004.

(51) Int. Cl. A01H 5/00; C07H 21/04; C12N 15/63; C12N 15/00; C12N 5/14

(52) U.S. Cl. 800/298; 536/23.6; 536/24.5; 435/320.1; 435/468; 435/419; 536/23.2

(57) ABSTRACT

The present invention is directed to a novel gene, Oslrk1, that is in involved in plant development, including root expansion. Methods of influencing this development are also described, as are transformed cells and transgenic plants comprising the described sequences.

The present invention claims benefit of U.S. Provisional Patent Application No. 60/614,549, filed Oct. 1, 2004, the contents of which are hereby incorporated by reference in their entirety.

### FIELD OF THE INVENTION

The present invention relates generally to genetic engineering of plants, and, more particularly, to regulating root expansion in transformed plants and plant cells.

### BACKGROUND OF THE INVENTION

Throughout this application, various publications are referenced within parentheses. Disclosures of these publications in their entireties are hereby incorporated by reference into this application to more fully describe the state of the art to which this invention pertains. Full bibliographic citations for the references may be found listed immediately preceding the claims.

It has been suggested that the plant serine/threonine receptor-like kinases (RLKs) play a crucial role in signaling events at the cell surface (Hardie et al., 1998; Morris, et al., 2003; Shiu, et al., 2004). The molecular basis of cell response to a variety of external factors is an active area of investigation. (Morris and Walker, 2003). RLKs are the class of transmembrane proteins comprising a single transmembrane spanning domain, a ligand-binding extracellular domain and a cytoplasmic catalytic kinase domain (Shiu and Bleecker, 2001). In annotated genome of Arabidopsis thaliana, RLKs are represented by more than 600 genes, whereas in rice at least 1132 members have been established (Shiu et al., 2004). The kinase domain of plant RLKs has a monophyletic origin and belongs to the same gene family as Drosophila melanogaster Pelle and mammalian interleukin receptor-associated kinase (Becraft, 2002). There are two different ways of classification of RLKs, the first one being based on the structural feature of the extracellular domain, which is thought to act as a ligand binding site (McCarty and Chory, 2000) and the second one being based on the biological function and the role of RLK either in control of plant growth and development or plant-microbe interaction and defense response (Shiu et al., 2004). There are over 21 different classes of Arabidopsis RLK ectodomains. (Becraft, 2002). One of the described and partially characterized classes of RLKs is lectin-like receptor kinases (LRKs). (Herve et al., 1996; 1999; Nishiguchi, et al., 2002, Navarro-Gochicoa, 2003; Riou et al., 2002; He et al., 2004; Shiu et al., 2004).

LRKs possess lectin-like extracellular domains, which share significant homology to legume-like lectins. Lectins are known as carbohydrate binding proteins without enzymatic activity towards recognized sugar (Loris, 2002). These are a structurally and evolutionarily diversified group of proteins. Lectins can be found in all kingdoms of life. Van Damme, 1998). Legume lectins refer to the plant lectins found exclusively in Leguminoseae. Most of these lectins isolated so far were identified from the mature seeds (Van Damme et al., 1998), and their concentration was very low in other vegetative organs. Legume lectins were strikingly similar in their primary, secondary and tertiary structures but, despite these similarities, showed considerable differences in their quaternary association and modes of monomer organization (Rudiger and Gabius, 2001). Interestingly, small alterations in tertiary structure as a result of variation in primary sequence lead to significant difference in quaternary structure (Vijayan and Chandra, 1999). Typically, specificity to glucose, N-acetyl-D-glucosamine, mannose, galactose or N-acetyl-D-galactosamine, L-fucose and complex type oligosaccharides had been demonstrated (Rudiger and Gabius, 2001). The physiological roles of legume lectins are still unclear, although they are storage proteins proposed to be involved in plant defense.

Lectin-like receptor kinase (LRKs) is a subclass of RLKs first described in Arabidopsis thaliana (Herve et al., 1996). At present, 103 members have been annotated in the rice genome (Shiu et al., 2004), with at least 42 LRKs identified in Arabidopsis (Barre et al., 2002), 9 members in Medicago truncatula (Navarro-Gochicoa et al, 2003) and several members in the Lombardy poplar (Nishiguchi et al, 2002). RLKs with legume-like lectin domains were not found in the complete genome sequences of yeast and human (Navarro-Gochicoa et al., 2003). They might be plant specific. The LRKs were shown to be expressed in different organs including the roots, mature leaves, stem, flowers and siliques. The physiological role of these proteins remains speculative. The presence of the legume-like lectin as receptor in LRKs suggested their role in perception of oligosaccharide mediated signals, but till now there is no evidence that LRKs possess an active receptor domain and sugar molecules might bind to it, whereas the kinase domain was found to be capable of autophosphorylation (Nishiguchi et al., 2002; He et al., 2004).

There remains a need in the art for a greater understanding of mechanisms of plant development, including root expansion, for methods of regulating this development, and for plants and plant cells in which such development can be regulated.

### SUMMARY OF THE INVENTION

The present invention relates to a novel lectin-like receptor kinase gene, referred to as Oryza sativa lectin-like receptor kinase 1 (Oslrk1), that plays a role in plant development. Results suggest that Oslrk1 responds to signals from sugars and/or phytohormones and is a negative regulator of certain aspects of plant development, including root expansion.

In particular, the invention provides an isolated nucleic acid which comprises (a) the nucleotide sequence set forth in SEQ ID NO: 1, (b) a nucleotide sequence that encodes the amino acid sequence of SEQ ID NO: 2, or (c) an antisense nucleotide sequence corresponding to a nucleotide sequence of (a) or (b). The present invention also provides vectors comprising the nucleic acid described above, operably linked to a promoter that controls expression in a plant cell. The present invention also provides methods for negative regulation of development in a plant, which include transforming plant cells with the nucleic acids described herein and cultivating the cells into plants.

In an aspect, the invention provides a method for promoting increased root growth in a plant, the method comprising transforming a plant cell with at least one nucleotide sequence operably linked to a promoter, wherein the nucleotide sequence is (a) an antisense nucleotide sequence corresponding to the nucleotide sequence set forth in SEQ ID NO: 1 or (b) an antisense nucleotide sequence corresponding to a nucleotide sequence that encodes the amino acid sequence of SEQ ID NO: 2, and cultivating the cell into a plant.

Furthermore, the invention provides an Oslrk1 Ds insertion mutant with a distinct phenotype of roots and aerial parts. Moreover, the invention provides a transgenic plant whose genome comprises a disruption of the Oslrk1 gene, wherein the disruption comprises the Ds insertion, and wherein the disruption results in the transgenic plant exhibiting increased root growth as compared to a wild-type plant.

The present invention further provides transformed plant cells and transgenic plants having incorporated into their genomes the nucleic acids described herein, as well as seeds of the transgenic plants. The present invention additionally provides nucleotide sequences that are homologous to the sequences described herein and which retain the biological activity of the described sequences, as well as methods, transformed cells, transgenic plants and seeds which use or incorporate these homologous sequences.

### BRIEF DESCRIPTION OF THE FIGURES

FIG. 1. The Phenotype Exhibited by the Oslrk1 Mutant. A. Five days old WT (left) and the mutant (right) seedlings grown in MS medium. B. and C. Roots of two weeks old WT and mutant seedlings, respectively, grown in MS medium. Roots of two months old WT (D) and mutant (E) plants grown in soil, F. Leaf morphology of two months old WT (left) and mutant (right) plants. Morphology of 45 days old WT (G) and mutant (H) plants where mutants displayed the delay of flowering (I). Three months old mutant plants (right) exhibited overall stronger phenotype of the aerial part compared to the WT. Panicles of mature WT (J) and mutant (K) plants showing more branches in mutant.

FIG. 2: Expression level of the Oslrk1 gene. The figure shows Northern hybridization analysis of Oslrk1 gene expression in WT and homozygous Oslrk1 mutant. Total RNA (10 ug) was extracted either from wt or mutant root tissues of two week old seedlings. Membranes were hybridized with DIG-labeled probe corresponding to 3′ UTR of the Oslrk1 gene. Transcript of 2.7 Kb in size was detected in WT plants and was absent in mutant root tissues (top panel). The bottom panel shows the amount of total RNA loaded (band obtained by hybridization of the stripped blot with probe corresponding to rice actin gene (RAct1).

FIG. 3: cDNA sequence of Oslrk1 (SEQ ID NO: 1).

FIG. 4: Domain and motif organization of OsLRK1 protein (SEQ ID NO: 2). Legume-like lectin domain is boxed and shown in FIG. 5 for lectin-domain amino acid sequence alignment. β-chain and α-chain, respectively, of lectin domain appear in bold within the box. The potential N-glycosylation sites are underlined. Signal peptide and transmembrane domains appear in italics. Serine/threonine kinase domain is presented in shading and the most conservative amino acids of 12 subdomains are shown in black bold font. Leucine residues in subdomain X (L⁵⁹⁹-L⁶⁰⁵-L⁶¹⁹-L⁶²⁶) that might be involved in leucine-zipper structure are framed. ATP-binding region (Leu³⁸⁷-Lys⁴¹³) in kinase domain is underlined.

FIG. 5. Comparison of aligned amino acid sequences of lectin-like domain of OsLR1 related sequences. Alignment was performed using TCoffee program (Notredame, C. et al., 2000). Identical amino acids are shaded and homologous sequences are framed. The asterisks indicate Ca²⁺ and crosses Mn²⁺-binding amino acids. A potential cleavage site (NDT) between β- and α-chains is framed and shaded. The OsLRK2 is the closest homolog to OsLRK1; P. nigra, lectin-receptor kinase from Populus nigra (AB030083 in DDBJ); MtLeRK1, expressed in roots lectin-receptor kinase from Medicago trunculata (AY358030); F.bean, mannose/glucose specific lectin from Field bean (P38662); B. purpurea, N-acetyl-D-galactose specific lectin from Bauhinia purpurea (P16030); L. sphaericus, lectin from Lathyrus sphaericus (16349); P. sativum, mannose-specific lectin from seeds of Pisum sativum (P02867); M. hemaglutinin and M. hemaglutinin, lectins from Maackia amurensis (1DBN_B (PDB) and 210523A); P. angolensis, lectin from Pterocarpus angolensis (Q8Q-A in PDB); R. pseudoacacia, bark lectin from Robinia pseudoacacia (BBA04604); AthLecRK1, lecin-like receptor kinase from Arabidopsis thaliana (AAB58725). NCBI GeneBank accession numbers and PBD ID are given in parentheses.

FIG. 6. The Expression Studies of the Oslrk1 Gene by RT-PCR (A) and Northern-blot hybridization (B). For RT-PCR about 2 ug of total RNA was added to PCR mix and primers corresponding to 5′UTR and 3′UTR sequences specific for the Oslrk1. The expected length of amplified transcript of the Oslrk1 (2.7 kb) is indicated with arrow (on the left side). Transcript of 500 bp of rice Actin 1 gene was amplified at the same time as internal control. For Northern blots total RNA (10 ug) isolated from different tissues (indicated above) as a template and DIG-labeled 3′UTR of the Oslrk1 as a probe were used. The transcript of 2.7 kb corresponded to the Oslrk1 gene (as indicated on the left side). The 18S rRNA was used as a quantitative control for total RNA.

FIG. 7. The Expression Analysis Transgenic Plants Harboring the Oslrk1 Promoter with Either GusA or Eyfp Genes. A-D. The expression of the Oslrk1 promoter::gusA in transgenic plants; E,G. The expression of EYFP in the Oslrk1 promoter::eyfp transgenic plants. (A). The GUS expression in adventitious roots of four day-old transgenic seedling and in adventitious and lateral roots of two-week seedlings (B); A cross sections of GUS stained roots showing expression at distal elongation zone (C) and mature zone where the bud of emerging lateral root is shown (D). In (C) Ep-Epidermis; Cx-Cortex; En-Endodermis; and Pc-Pericuycle cells. (E,G). The micrographs of EYFP expression in roots of transgenic plants; (E,G). The confocal images showing EYFP expression in the vascular cells of the roots, (F). The visible light image of the root; (G). The merged images of E and F.

FIG. 8. Regulation of the Oslrk1 Transcript with Different Hormones, SA (A) and Sugars (B). In all experiments DIG-labeled probe corresponding to 3′UTR of the Oslrk1 gene was utilized in Northern-blot hybridization. As a quantitative control of loaded total RNA, filters used for the Northern-hybridization with the Oslrk1 probe were washed and re-hybridized with DIG-labeled probe corresponding to the rice Actin 1 gene. The ratio on the top of the Northern blots indicated the actual level of expression of the Oslrk1 in the samples and it was calculated as ratio of intensity of hybridization signal obtained with the Oslrk1 specific probe to the signal intensity obtained with Act1 probe.

FIG. 9. The treatment of WT and mutant seedlings with MeJA. The seeds of WT and mutant were germinated and grown in MS (with 1% of Suc added) containing different concentrations of MeJa (1 μM, 5 μM and 10 μM) along with control (A). The Bar charts represent length and number of adventitious roots as well as number of lateral roots (B). Around 30-50 seedlings were measured for the each data points. The p-Value was calculated for the Number of adventitious roots (0.005), Length of adventitious roots (0.00) and Number of lateral roots (0.19).

FIG. 10. The Differential Response of the Oslrk1 Mutant to Man (A and B) and Gal (C and D). Seeds of WT and mutant plants were germinated at different concentration of Gal/Man for 1 week in MS medium. A and C shows the response of the seedlings germinated in Man and Gal, respectively. Bar charts representing the shoot length, number of length of adventitious roots and germination rate of WT and the Oslrk1 mutant seedlings to applied Man (B) and Gal (D). B and D represent the average of three independent experiments. For each experiment at least 50 seedlings were germinated in appropriate monosaccharides and the data points were measured. The p-Values for mannose treatment (C) on the shoot length (0.453), number (0.509) and length (0.527) of adventitious roots was obtained. For galacatose treatment (D) the p-Values were 0.00, 0.01, and 0.00 for number and length of adventitious roots and shoot length, respectively.

FIG. 11. Proposed Model for the Role of the OsLRK1 as a Negative Regulator of Rice Root Growth.

FIG. 12. Expression Study of Oslrks-Northern blots. YL-young leaves; ML-mature leaves. Shows the mRNA expression results by northern blot using different Irks as probe.

FIG. 13. Expression Analysis of Oslrks Under Sugar Treatment.

FIG. 14. Expression Analysis of Oslrks Under Hormone treatment.

FIG. 15. Expression Study of Oslrks Under Abiotic Stress Treatments.

### DETAILED DESCRIPTION OF THE INVENTION

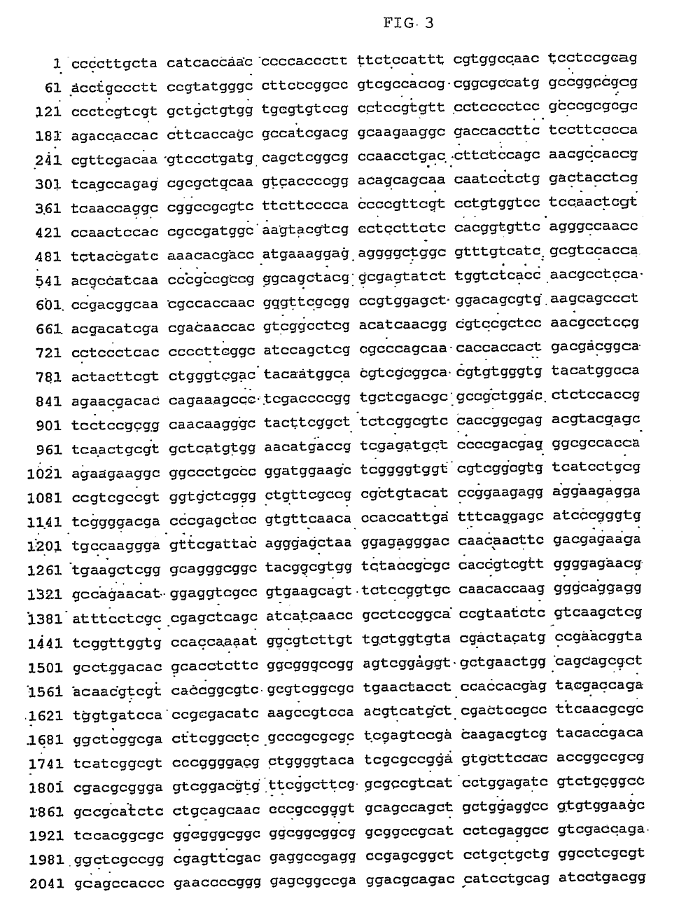

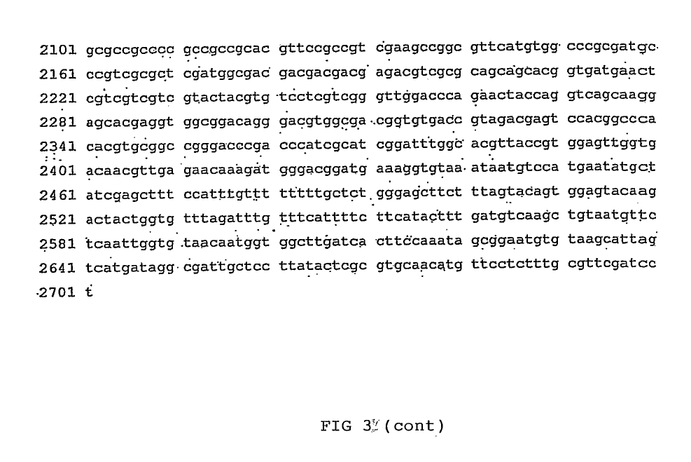

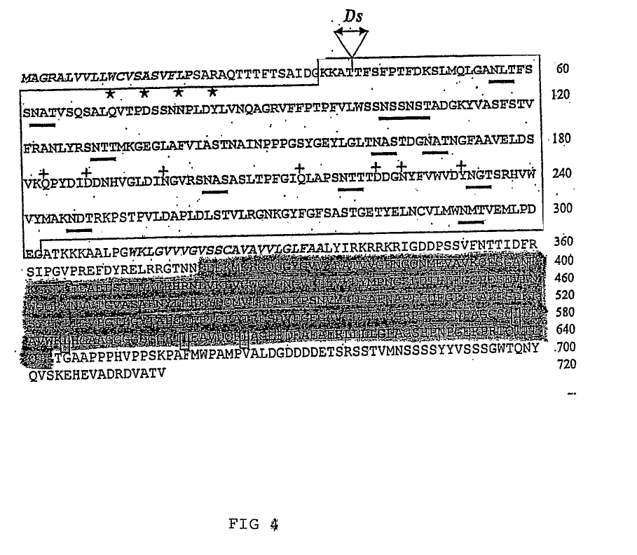

A new gene involved in plant development, Oslrk1, is described. The cloned cDNA is 2701 bp in length (FIG. 3) (SEQ ID NO: 1). The putative OSLRK1 protein (SEQ ID NO: 2) is shown in FIG. 4.

Moreover, a rice lectin-like receptor kinase 1 (Oslrk1) mutant was isolated from a visual phenotype screen of several hundred Ds insertion lines and characterized. Mutant plants exhibited an expanded root system with more adventitious and longer lateral roots. The aerial part of the plant showed larger leaves, delayed flowering, and a higher seed yield compared with wild type. The mutant segregated the phenotype with Basta resistance and bar genotype indicating that the phenotype was linked to Ds insertion. The Oslrk1 Ds insertion line was a null mutant as no transcript was detected in homozygous mutant.

One of the advantages of the Ac/Ds transposon system is the ability to obtain a revertant of the mutant phenotype caused by Ds insertion by remobilization of the Ds element in the presence of transposase source (Ramachandran and Sundaresan, 2001). A homozygous revertant of the Oslrk1 mutant was obtained, which rescued the mutant phenotype to the WT. The generation of revertant confirmed that the mutant phenotype observed was due to the knockout of the Oslrk1 gene by the Ds insertion.

The Ds insertion was located 117 bp downstream of ATG codon, in the 1st exon, and resulted in a loss of the Oslrk1 transcript as confirmed by Northern analysis. The Oslrk1 gene belongs to a multi-gene family comprising of at least 64 members in the annotated rice genome. Revertant was obtained by crossing homozygous mutant line with plants harboring Ac transposase. The Northern-blot hybridization, histochemical and fluorescence analysis of transgenic plants harboring the Oslrk1 promoter::gusA and eyfp constructs, respectively, showed that the Oslrk1 gene is predominantly expressed in the vasculature of adventitious and lateral roots, but not in the root cap. The transcript of the Oslrk1 was induced in response to methyl jasmonate (MeJA), but repressed by gibberellic acid (GA) and salicylic acid (SA). In addition to this, the Oslrk1 gene was down regulated by several sugars tested. Furthermore, the mutant plants displayed hypersensitivity to galactose and reduced sensitivity to MeJA treatment. These results suggest that OsLRK1 plays a role as negative regulator of certain aspects of plant development, particularly root expansion, and responds to signals from sugars and phytohormones.

An embodiment of the present invention thus provides an isolated nucleic acid which comprises (a) the nucleotide sequence set forth in SEQ ID NO: 1, (b) a nucleotide sequence that encodes the amino acid sequence of SEQ ID NO: 2; and/or (c) an antisense nucleotide sequence corresponding to the nucleotide sequence of (a) or (b). The nucleic acid may be DNA or RNA, and can be cDNA, genomic DNA, or mRNA. In one embodiment, the nucleic acid is a fusion gene, such as an Oslrk1-GUS fusion gene or an Oslrk1-EYFP fusion gene. The nucleic acid can be a transcriptional fusion such as Oslrk1 promoter::gusA or Oslrk1 promoter::eyfp. The present invention also provides a vector comprising the nucleic acid described above, operably linked to a promoter that controls expression in a plant cell. The vector may be a plant expression vector or a vector useful for plant transformation. Any suitable vector well known to skilled artisans may be utilized. The promoter may be any promoter useful for expression of genes in plants. Suitable promoters are well known to those of ordinary skill in the art. Preferred promoters include the Maize ubiquitin promoter and the CaMV 35S promoter. As is known in the art, promoters can include inducible and/or repressible promoters and enhancers such that the expression of the nucleic acid and encoded polypeptide can be regulated based on various physiological conditions and signals. The nucleic acids of the invention can express the described polypeptides both in vivo and in vitro using various techniques well known to those of skill in the art, including, for example, transduction, transfection or transformation of the nucleic acid or vector into a cell and in vitro transcription and translation.

The invention further provides a method for negative regulation of development in a plant, the method comprising transforming a plant cell with at least one nucleotide sequence operably linked to a promoter, wherein the nucleotide sequence is (a) a nucleic acid comprising the nucleotide sequence set forth in SEQ ID NO: 1, or (b) a nucleic acid comprising a nucleotide sequence that encodes the amino acid sequence of SEQ ID NO: 2, and cultivating the cell into a plant. Cultivating the cell into a plant can involve any technique, including those described herein and others known in the art, for growing or continuing to grow a plant cell to a mature plant. The promoter may be any suitable promoter and the plant may be any plant species, preferably a monocot, as described herein. Examples of preferred promoters include the Maize ubiquitin promoter and the CaMV 35S promoter. Examples of plant species include maize, wheat, barley, rye, and others.

In the methods described herein, plant development encompasses all aspects of the developmental control of morphogenesis, including the coordination of cell growth, cell division and cell differentiation and the reflection of this coordination as seen in organ growth and/or resulting overall plant growth. The sequences of the present invention can be introduced into any plant of interest using techniques well known to skilled artisans, and can be used to transform any plant species using techniques well known to skilled artisans. The sequences to be introduced can be contained in expression cassettes for expression in the particular plant of interest.

In a preferred embodiment, the development is root expansion. The regulation can comprise transmitting signals from sugar and/or a phytohormone. The invention further provides a method for promoting increased root growth in a plant, the method comprising transforming a plant cell with at least one nucleotide sequence operably linked to a promoter, wherein the nucleotide sequence is (a) an antisense nucleotide sequence corresponding to the nucleotide sequence set forth in SEQ ID NO: 1 or (b) an antisense nucleotide sequence corresponding to a nucleotide sequence that encodes the amino acid sequence of SEQ ID NO: 2, and cultivating the cell into a plant. The promoter may be any suitable plant promoter and is preferably maize ubiquitin or CaMV 35S. The plant may be any species and is preferably a monocot. Examples of suitable plant species include maize, wheat, barley, rye, and others.

In a preferred embodiment, the increased root growth comprises an expanded root system. In preferred embodiments, the expanded root system includes more adventitious and/or longer lateral roots.

The present invention also provides a transformed plant cell having stably incorporated into its genome at least one nucleotide sequence operably linked to a promoter, wherein the nucleotide sequence is (a) a nucleic acid comprising the nucleotide sequence set forth in SEQ ID NO: 1, (b) a nucleic acid comprising a nucleotide sequence that encodes the amino acid sequence of SEQ ID NO: 2, or (c) a nucleic acid comprising an antisense nucleotide sequence corresponding to a nucleotide sequence of (a) or (b). The promoter can be any suitable promoter. The cell can be of any plant type and any species, and is preferably from a monocot.

The present invention further provides a transgenic plant having stably incorporated into its genome at least one nucleotide sequence operably linked to a promoter, wherein the nucleotide sequence is (a) a nucleic acid having the nucleotide sequence set forth in SEQ ID NO: 1, (b) a nucleic acid having a nucleotide sequence that encodes the amino acid sequence of SEQ ID NO: 2, or (c) a nucleic acid having an antisense nucleotide sequence corresponding to a nucleotide sequence of (a) or (b), The promoter can be any suitable promoter. The plant can be any plant species and is preferably a monocot. The invention also provides seeds of the transgenic plants described herein.

The invention further provides a method for regulating development in a plant, the method comprising transforming a plant cell with at least one nucleotide sequence operably linked to a promoter, wherein the nucleotide sequence is (a) a nucleic acid comprising the nucleotide sequence set forth in SEQ ID NO: 1 (b) a nucleic acid comprising a nucleotide sequence that encodes the amino acid sequence of SEQ ID NO: 2 or (c) a nucleic acid comprising an antisense nucleotide sequence corresponding to a nucleotide sequence of (a) or (b), and cultivating the cell into a plant. The promoter can be any suitable promoter. The plant can be any plant species and is preferably a monocot. In a preferred embodiment, the development comprises root growth. In another preferred embodiment, the regulating comprises introducing a modification in an aerial part of the plant.

The present invention further provides a nucleotide sequence having greater than 50% homology to a full-length nucleotide sequence set forth in SEQ ID NO: 1, wherein the homologous nucleotide sequence encodes a polypeptide which retains biological activity of the full length sequence, namely the regulating development activities described herein.

The homology of the nucleotide sequence is preferably about 80%, and more preferably, about 95%.

The present invention further provides a method for negative regulation of root expansion in a plant, said method comprising transforming a plant cell with at least one nucleotide sequence operably linked to a promoter, wherein the nucleotide sequence is (a) a nucleotide sequence having greater than 50% homology to a full-length nucleotide sequence set forth in SEQ ID NO: 1, wherein the homologous nucleotide sequence encodes a polypeptide which retains biological activity of the full length sequence, or (b) a nucleotide sequence having greater than 50% homology to a nucleotide sequence that encodes the amino acid sequence of SEQ ID NO: 2, wherein the nucleotide sequence encodes a polypeptide which retains biological activity of the full length sequence, and cultivating the cell into a plant. The homology of the nucleotide sequence is preferably about 80%, and more preferably, about 95%.

The invention further provides a method of promoting root expansion in a plant, the method comprising transforming a plant cell with at least one nucleotide sequence operably linked to a promoter, wherein the nucleotide sequence is (a) an antisense nucleotide sequence corresponding to a nucleotide sequence having greater than 50% homology to the nucleotide sequence set forth in SEQ ID NO: 1, or (b) an antisense nucleotide sequence corresponding to a nucleotide sequence having greater than 50% homology to a nucleotide sequence that encodes the amino acid sequence of SEQ ID NO: 2, and cultivating the cell into a plant. The homology of the nucleotide sequence is preferably about 80%, and more preferably, about 95%.

The present invention further provides a transformed plant cell having stably incorporated into its genome at least one nucleotide sequence operably linked to a promoter, wherein the nucleotide sequence is (a) a nucleotide sequence having greater than 50% homology to a full-length nucleotide sequence set forth in SEQ ID NO: 1, wherein the homologous nucleotide sequence encodes a polypeptide which retains biological activity of the full length sequence, (b) a nucleotide sequence having greater than 50% homology to a nucleotide sequence that encodes the amino acid sequence of SEQ ID NO: 2, wherein the nucleotide sequence encodes a polypeptide which retains biological activity of the full length sequence, or (c) an antisense nucleotide sequence corresponding to a nucleotide sequence of (a) or (b). The homology of the nucleotide sequence is preferably about 80%, and more preferably, about 95%.

The present invention further provides a transgenic plant having stably incorporated into its genome at least one nucleotide sequence operably linked to a promoter, wherein the nucleotide sequence is (a) a nucleotide sequence having greater than 50% homology to a full-length nucleotide sequence set forth in SEQ ID NO: 1, wherein the homologous nucleotide sequence encodes a polypeptide which retains biological activity of the full length sequence, (b) a nucleotide sequence having greater than 50% homology to a nucleotide sequence that encodes the amino acid sequence of SEQ ID NO: 2, wherein the nucleotide sequence encodes a polypeptide which retains biological activity of the full length sequence, or (c) an antisense nucleotide sequence corresponding to a nucleotide sequence of (a) or (b). The homology of the nucleotide sequence is preferably about 80%, and more preferably, about 95%. The invention also provides a seed of this plant.

The present invention also provides a transgenic plant whose genome comprises a disruption of the Oryza sativa Lectin-Like Receptor Kinase 1 (Oslrk1) gene, wherein the disruption comprises a Ds insertion, and wherein the disruption results in the transgenic plant exhibiting increased root growth as compared to a wild-type plant. In a preferred embodiment, the disruption is a homozygous disruption. In a preferred embodiment, the disruption is a complete knockout of the Oslrk1 gene. In another preferred embodiment, the Ds insertion is located 117 bp downstream of the ATG codon in the first exon of the Oslrk1 gene. In another embodiment, the increased root growth is an expanded root system. In another embodiment, the expanded root system includes more adventitious or longer lateral roots.

In an embodiment, the transgenic plant exhibits modifications in its aerial part. The modifications can be larger leaves, delayed flowering, or higher seed yield as compared to a wild-type plant. In an embodiment, the higher seed yield is a yield that is about 20% higher than that of a wild-type plant.

In another embodiment, the transgenic plant exhibits a hypersensitivity to D-galactose as compared to that of a wild-type plant. In a still further embodiment, the flowering is delayed by about five to about seven days as compared to the flowering in a wild-type plant. The transgenic plant of the present invention, when mature, can be about 30% taller than a wild-type plant grown in similar conditions. The transgenic plant, when mature, can exhibit about 70% more branches in its panicles than a wild-type plant grown in similar conditions. The transgenic plant, at the seedling stage, can exhibit a shoot length of about two times that of a wild-type plant grown in similar conditions. In another embodiment, the transgenic plant, at the seedling stage, can exhibit about 56% more adventitious lateral roots than a wild-type plant grown in similar conditions. In a further embodiment, the transgenic plant, at the seedling stage, exhibits lateral roots that are about 74% longer than lateral roots of a wild-type plant grown in similar conditions. The transgenic plant can be any plant species, and is preferably a monocot. Preferred species include maize, wheat, barley, rye, and others. The invention further provides a seed of the transgenic plant.

The invention further provides an isolated nucleic acid comprising an Oslrk1 gene, wherein the gene comprises a Ds insertion located about 117 bp downstream of the ATG codon in its first exon.

The invention also provides a method for increasing root growth in a plant, the method comprising manipulating the genome of a plant cell to comprise a disruption of the Oslrk1 gene, wherein the disruption comprises a Ds insertion, and cultivating the cell into a plant. In a preferred embodiment, the disruption is a complete knockout of the Oslrk1 gene. In a preferred embodiment, the disruption is a homozygous disruption. In an embodiment, the increased root growth comprises an expanded root system. In an embodiment, the expanded root system includes more adventitious and/or longer lateral roots. In a preferred embodiment, the Ds insertion is located 117 bp downstream of the ATG codon in the first exon of the Oslrk1 gene.

The present invention further provides a method of modifying the aerial part of a plant, the method comprising manipulating the genome of a plant cell to comprise a disruption of the Oslrk1 gene, wherein the disruption comprises a Ds insertion, and cultivating the cell into a plant. In a preferred embodiment, the disruption is a complete knockout of the Oslrk1 gene. In a preferred embodiment, the disruption is a homozygous disruption. In an embodiment, the modifications in the aerial part of the plant are larger leaves, delayed flowering, or higher seed yield as compared to a wild-type plant. The higher seed yield can be a yield that is about 21% higher than that of a wild-type plant.

The invention further provides a method for increasing sensitivity to D-galactose in a plant, the method comprising manipulating the genome of a plant cell to comprise a disruption of the Oslrk1 gene, wherein the disruption comprises a Ds insertion, and cultivating the cell into a plant. In a preferred embodiment, the Ds insertion is located 117 bp downstream of the ATG codon in the first exon of the Oslrk1 gene. In another preferred embodiment, the disruption is a complete knockout of the Oslrk1 gene. In a preferred embodiment, the disruption is a homozygous disruption.

The invention further provides an isolated Ds insertion mutant of the Oslrk1 gene, wherein the Ds insertion is located 117 bp downstream of the ATG codon in the first exon of the Oslrk1 gene.

As used herein, an isolated or purified nucleic acid or protein, or biologically active portion thereof, is substantially free of other cellular material, or culture medium when produced by recombinant techniques, or substantially free of chemical precursors or other chemicals when chemically synthesized. Preferably, an isolated nucleic acid is free of sequences (preferably protein encoding sequences) that naturally flank the nucleic acid (i.e. sequences located at the 5′ and 3′ ends of the nucleic acid) in the genomic DNA of the organism from which the nucleic acid is derived.

The polynucleotide or nucleic acid compositions of the present invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. The polynucleotides of the invention may be isolated or substantially pure.

Recombinant constructs comprising the OsLRK1 gene, or a Ds insertion mutant thereof, may be capable of replicating autonomously in a host cell. Alternatively, the recombinant construct may become integrated into the chromosomal DNA of the host cell. Such a recombinant polynucleotide comprises a polynucleotide of genomic, cDNA, semi-synthetic, or synthetic origin which, by virtue of its origin or manipulation, 1) is not associated with all or a portion of a polynucleotide with which it is associated in nature; 2) is linked to a polynucleotide other than that to which it is linked in nature; or 3) does not occur in nature.

Therefore, recombinant nucleic acids comprising sequences otherwise not naturally occurring are additionally provided by this invention. Although the described sequences may be employed, they will often be altered, e.g., by deletion, substitution or insertion.

Protein modifications or fragments are provided by the present invention for wildtype and mutant OsLRK1 polypeptides or fragments thereof which are substantially homologous to primary structural sequence but which include, for example, in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by persons of ordinary skill in the art. A variety of methods for labeling polypeptides and a variety of substituents or labels useful for such purposes are well known by persons of ordinary skill in the art, and include radioactive isotopes such as ³²P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation.

As described, in addition to substantially full-length proteins, the present invention provides for biologically active fragments and homologs of the polypeptides. The term “polypeptide” as used herein refers to both a full length protein and a portion of the protein as a polypeptide fragment.

The present invention also provides for fusion polypeptides, comprising OsLRK1 polypeptides and fragments thereof and polypeptides or fragments of other proteins as known in the art. Homologous polypeptides may be fusions between two or more polypeptide sequences or between the sequences of OsLRK1 and a related protein. Likewise, heterologous fusions may be constructed which would exhibit a combination of properties or activities of the derivative proteins. For example, ligand-binding or other domains may be “swapped” between different new fusion polypeptides or fragments. Such homologous or heterologous fusion polypeptides may display, for example, altered strength or specificity of binding and may include for example partners such as immunoglobulins, bacterial C-galactosidase, trpE, protein A, C-lactamase, alpha amylase, alcohol dehydrogenase and yeast alpha mating factor.

Fusion proteins will typically be made by either recombinant nucleic acid methods, as described below, or may be chemically synthesized. Techniques for the synthesis of polypeptides are well known by persons of ordinary skill in the art.

Other protein modifications include amino acid substitution. Substitutional variants typically contain the exchange of one amino acid for another at one or more sites within the protein, and may be designed to modulate one or more properties of the polypeptide, such as stability against proteolytic cleavage, without the loss of other functions or properties. Amino acid substitutions may be made on the basis of similarity in polarity, charge, solubility, hydrophobicity, hydrophilicity, and/or the amphipathic nature of the residues involved. Preferred substitutions are ones which are conservative, that is, one amino acid is replaced with one of similar shape and charge. Conservative substitutions are well known to persons of ordinary skill in the art and typically include, though not exclusively, substitutions within the following groups: glycine, alanine; valine, isoleucine, leucine; aspartic acid, glutamic acid; asparagine, glutamine; serine, threonine; lysine, arginine; and tyrosine, phenylalanine.

Certain amino acids may be substituted for other amino acids in a protein structure without appreciable loss of interactive binding capacity with structures such as, for example, antigen-binding regions of antibodies or binding sites on substrate molecules or binding sites on proteins interacting with an polypeptide. Since it is the interactive capacity and nature of a protein which defines that protein's biological functional activity, certain amino acid substitutions can be made in a protein sequence, and its underlying DNA coding sequence, and nevertheless obtain a protein with like properties. In making such changes, the hydropathic index of amino acids may be considered. The importance of the hydrophobic amino acid index in conferring interactive biological function on a protein is generally understood in the art. Alternatively, the substitution of like amino acids can be made effectively on the basis of hydrophilicity. The importance of hydrophilicity in conferring interactive biological function of a protein is generally understood in the art (See e.g. U.S. Pat. No. 4,554,101). The use of the hydrophobic index or hydrophilicity in designing polypeptides is further discussed in U.S. Pat. No. 5,691,198. These patents are hereby incorporated by reference in their entireties.

A recombinant nucleic acid is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques. This phrase is also meant to encompass a gene which is removed from its normal regulatory expression constraints, as in the case where a gene product is overexpressed due to the presence of multiple copies of the gene or up regulated promoter or enhancer signals, increased mRNA or protein half life and the like.

“Regulatory sequences” refers to those sequences which affect the expression of the gene (including transcription of the gene, and translation, splicing, stability or the like of the messenger RNA).

Large amounts of the polynucleotides of the present invention may be produced by a suitable host cell transformed with a nucleotide sequence described herein. Natural or synthetic polynucleotide fragments coding for the peptide or a desired fragment can be incorporated into recombinant polynucleotide constructs (vectors), usually DNA constructs, capable of introduction into and replication in a prokaryotic or eukaryotic cell. Typically, the vectors will be suitable for replication in a unicellular host, such as yeast or bacteria, but may also be intended for introduction to (with and without integration within the genome) cultured mammalian or plant or other eukaryotic cell lines. The most commonly used prokaryotic hosts are strains of Escherichia coli, although other prokaryotes, such as Bacillus subtilis or Pseudomonas may also be used. Mammalian or other eukaryotic host cells, such as those of yeast, filamentous fungi, plant, insect, or amphibian or avian species, may also be useful for production of the proteins of the present invention. As is well known in the relevant art, regulating polynucleotide expression can result in regulation of polypeptides encoded by the polynucleotide.

Expression and cloning vectors preferably contain a selectable marker gene. Typical marker genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of an appropriate selectable marker will depend on the host cell, and appropriate markers for different hosts are well known to persons of ordinary skill in the art.

The vectors containing the nucleic acids of interest can be transcribed in vitro, and the resulting RNA introduced into the host cell by well-known methods, e.g., by injection, or the vectors can be introduced directly into host cells by methods well known to persons of ordinary skill in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation.” The cells into which have been introduced nucleic acids described above are meant to also include the progeny of such cells.

Clones are selected by using markers, depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

Prokaryotic or eukaryotic cells transformed with the polynucleotides of the present invention are useful not only for the production of the nucleic acids and polypeptides of the present invention, but also, for example, in studying the characteristics of OsLRK polypeptides. Plant cells transformed with the polynucleotides of the present invention are useful also for growing plants expressing the polynucleotides and polypeptides of the present invention. The nucleotides of the present invention can also be transformed into plants that have already undergone some growth.

A polynucleotide of the present invention can be expressed in either sense or antisense orientation, as desired. It will be appreciated that control of gene expression in either sense or antisense orientation can have a direct impact on the observable plant characteristics. Antisense technology, known in the art, can be conveniently used for gene expression in plants. To accomplish this, a nucleic acid segment from the desired gene is cloned and operably linked to a promoter such that the antisense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest.

Insertional mutagenesis with T-DNA and transposons (such as AC/Ds, Spm/En) represents a powerful tool in functional genomics as it mediates gene knock-out and can result in a mutant phenotype. (Ramachandran, S., and Sundaresan, V. (2001)). Despite the simplicity of using T-DNA mutagenesis, transposable elements offer several advantages, such as single insertion in the genome and the possibility of rescuing the inactivated gene by remobilization of the transposable element.

Other techniques known in the art, such as miRNA, RNAi, and sRNA lead to gene inactivation by degradation of its transcript. (Mallory, A., and Vaucheret, H. (2004); Dawnward, J. (2004); Finnegan, B. and Matzke, M. (2003)). One of ordinary skill in the art would readily recognize from the teachings herein that such techniques are suitable for suppressing the expression of the Oslrk1 gene product in accordance with the present invention. Thus, the invention further provides a variety of methods of inactivating, disrupting, or otherwise blocking the activity or expression of the Oslrk1 gene or its products to achieve the characteristics of transgenic plants and transformed cells described herein.

In light of the preceding description, one of ordinary skill in the art can practice the invention to its fullest extent. The following examples, therefore, are merely illustrative and should not be construed to limit in any way the invention as set forth in the claims which follow.

### EXAMPLE 1

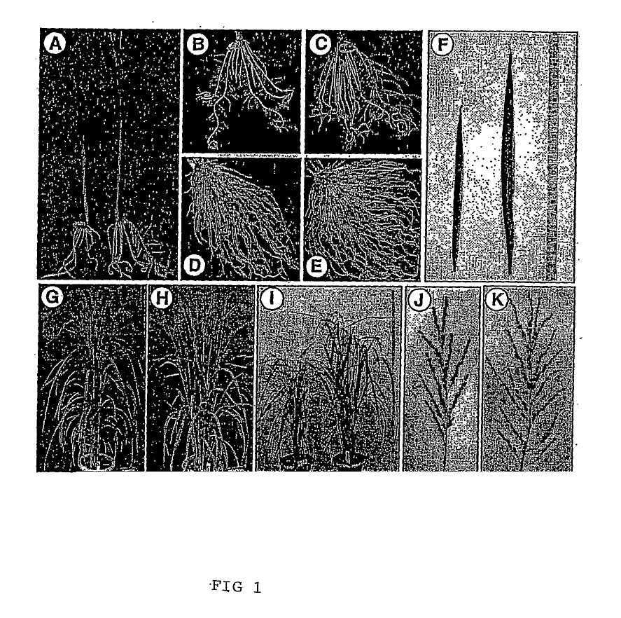

The Mutant Isolation and Phenotype Characterization. Several hundred F3/F4 gene trap rice Ds insertion lines, generated previously by our group (Kolesnik T. et. al., 2004), were subjected to visible phenotype screens. Among these transposants, a Basta resistant (Bar^{R}) line with an obvious phenotype was selected for further analysis. The mutant plants showed longer and broader leaves, increased number of panicle branches, and, as a result, higher seed yield than the wild-type (WT) plants (FIG. 1, Table 1). The mutant phenotype was stable in successive generations. Six- (FIG. 1A) and fourteen-day old (Table 1) mutant seedlings grown in MS media showed shoot length two times longer than WT seedlings grown in similar conditions (FIG. 1A, Table 1). At the seedling stage, mutant plants had 56% more adventitious and 74% longer lateral roots (FIG. 1B, Table 1).

TABLE 1
Phenotype characteristics of Oslrk1 mutant.

| | WT | | Oslrk1 Mutant | | p-Value |
|---|---|---|---|---|---|
| Shoot length (cm) | 16.2 | (±2.51) | 27.4 | (±4.45) | 0.000 |
| Leaf length (cm) | 36.5 | (±4.51) | 46.6 ± (3.95) | | 0.000 |
| Leaf width (cm) | 1.25 | (±0.09) | 1.65 | (±0.12) | 0.000 |
| No. of panicle branches | 7.0 | (±0.51) | 12.0 | (±0.71) | 0.000 |
| No. of seeds | 1056 | (±200.1) | 1275 | (±206.5) | 0.008 |
| No. adventitious roots | 6.3 | (±1.61) | 9.8 | (±1.95) | 0.000 |
| Lateral roots length (cm) | 0.78 | (±0.17) | 1.36 | (±0.31) | 0.244 |

Leaf width and length were measured from mature 2 month-old plants. Shoot length, number of adventitious roots, and lateral root length were determined for seedlings grown in MS agar medium in a growth chamber at 25° C. for two weeks. Approximately 50 plants were used for each data point. Standard Deviation Value (SDV) is given in parentheses. All the above parameters were subjected to t-test and the p-Values are provided. All were p < 0.05 and thus were significantly different.

The mutant plants continued to show similar differences throughout their development. The average length of the flag leaf of two month-old WT plants was 37 cm, whereas the mutant flag leaf showed an average of 47 cm (27% longer). A similar difference was observed in width of flag leaves of mutant plants (30% wider than that of WT flag leaf) (FIG. 1C and Table 1). Apart from these characteristics the Ds insertion line displayed a late flowering phenotype with five to seven days delay when compared with the WT plants (FIG. 1D). The mature mutant plants were about 30% taller (data not shown) than the WT plants grown in similar conditions (FIG. 1E). At the mature stage, the mutant plants showed approximately 70% more branches in the panicles (FIG. 1F, Table 1), resulting in 21% higher seed yield (Table 1). The height of the mutant plant, panicle branches and seed yield were statistically significant when compared to WT characteristics. At the mature stage, the mutant plants had more adventitious and longer lateral roots than the WT, similar to the differences observed at seedling stage (FIG. 1B).

In order to link mutation caused by Ds insertion to the mutant phenotype, the segregation analysis of bar gene (conferring resistance to the Basta herbicide), the selection marker, was performed. Approximately 200 F₃ seedlings derived from a heterozygous F₂ plant were sprayed with the Basta herbicide. A 3:1 ratio of Basta resistant (Bar^{R}) to sensitive (Bar^{S}) seedlings in the F₃ progeny was obtained which indicated that the mutant might possess a single Ds insertion. To confirm that these Basta resistant plants indeed harbored the bar gene, genomic DNA from the leaves of these plants was isolated and used as template for PCR analysis in combination with primers designed for the bar gene.

Analyzing segregation ratio is an important (but not sufficient) step in linking phenotype and mutation caused by Ds insertion. The phenotype could be due to a footprint in another gene(s) within the same genetic locus as a result of primary Ds transposition (Chin et al., 1999). In order to show that the Ds and the mutant phenotype co-segregate, in another series of experiments, without spraying Basta herbicide, genomic DNA extracted from 200 F₄ seedlings derived from of F₃ heterozygous plants were subjected to PCR analysis to check the presence of bar gene. Plants that were bar negative by PCR exhibited WT characteristics, whereas, plants possessing bar gene demonstrated growth parameters of the mutant, confirming the linkage between phenotype and mutation caused by Ds insertion.

### Molecular Characterization of the Mutant

### EXAMPLE 2

Gene Tagged by Ds Insertion Encoded Lectin-Receptor Kinase (OsLRK1). To determine the sequences flanked by Ds insertion, the genomic DNA isolated from the leaves of the mutant plants was subjected to TAIL-PCR (Liu and Whittier, 1995), followed by sequencing. Both 5′ and 3′ Ds flanking sequences were obtained and 8-nucleotide target duplication site (5′ TTCACCAG 3′) introduced by Ds element upon integration was obtained. The flanking sequences were submitted for sequence similarity searches using BLASTN and BLASTX alignment algorithms in TIGR (The Institute for Genome Research), RGP (Rice Genome Research Program) and NCBI (National Center for Biotechnology Information) public databases. The results revealed that the Ds tagged gene encoded protein homologous to lectin-receptor protein kinase and this gene was designated Oryza sativa lectin-receptor kinase 1, or “Oslrk1”.

### EXAMPLE 3

The Oslrk1 Mutant had a Single Copy of Ds Element. The segregation ratio (Bar^{R}: Bar^{S}) 3:1 indicated the presence of single copy of Ds element in the genome or multiple copies within the same genetic locus. The presence of two or more Ds elements would complicate the mutant phenotype and its characterization. In order to confirm the copy number of Ds elements, Southern-blot hybridization was performed using genomic DNA extracted from the leaves of mutant plants with either bar or gusA genes as probes (Methods). In both cases, a single band with each of the restriction enzymes used was obtained. These results confirmed the presence of a single Ds element in the mutant line (data not shown).

### EXAMPLE 4

Complete Knock-out of Oslrk1 by Ds Insertion. The Oslrk1 gene was located on Chromosome 2, BAC clone OJ1038_A06 (at 125.6 cM on the genetic map). The gene region of Oslrk1 is predicted to be 3,257 bp long with one intron (1 kb) whereas the open reading frame (ORF) was 2,224 bp long. The 5′ and 3′ untranslated regions (UTRs) were at least 107 bp and 392 bp long, respectively, as we have shown by RT-PCRs using different primer combinations designed based on assumed 5′UTR and 3′UTR sequences.

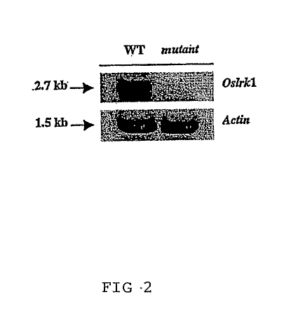

In order to find the precise location of Ds insertion within the Oslrk1 gene, PCR was performed using genomic DNA isolated from the mutant leaves and specific primers designed for 5′UTR of Oslrk1 gene and 5′ prime end of gusA gene. The PCR product was sequenced and the analysis of this sequence revealed that Ds insertion was located 117 bp downstream of ATG codon in the first exon. Further analysis by Northern-blot hybridization using total RNA purified from the WT, and homozygous mutant plants, was performed using 3′UTR of Oslrk1 gene as a probe (FIG. 2). Oslrk1 gene transcript was detected only in the WT but not in the homozygous mutant plants, suggesting a possible knockout of the gene.

### EXAMPLE 5

Re-mobilization of Ds Element from the Oslrk1 Gene Rescued the Mutant Phenotype. To rescue the mutant phenotype caused by the Ds insertion, 25 homozygous Oslrk1 mutant plants were crossed with homozygous plants harboring Ac transposase (Ac1 or Ac5; Kolesnik et al., 2004) to remobilize the Ds element. The R1 and majority of R2 plants showed growth characteristics of the mutant. The genomic DNA from transgenic plants and WT as a control were extracted and used as a template for PCR in combination with primers designed for the 5′ and 3′ sequences flanking the Ds element. The amplified fragments were sequenced and aligned using CLUSTALW program (Thompson et al., 1994), then analyzed for the presence of footprint due to Ds excision. We have obtained PCR products containing different footprints for the 22 R₂ plants, showing the remobilization of the Ds element. The length of the footprints (empty donor sites) varied from 8 nucleotides (indicating a perfect Ds element excision) to 2 nucleotides (as a result of deletion of several central nucleotides; Schiefelbein et al., 1988). Due to the fact that Ds element was inserted 117 bp (encoding 39 amino acids) downstream of ATG codon, in the first exon, 3 bp (or multiples of 3) footprints would result in proper translation of mRNA obtained after excision of Ds element. Only one plant with 3 bp footprint was obtained and it showed the growth parameters of the WT plants. However, plants with footprints different from 3 bp did not rescue the mutant phenotype.

### EXAMPLE 6

The Lectin-Like Kinases are Encoded by a Multi-Gene Family in Rice. Lectin-like kinases were shown to be encoded by multi-gene families in Arabidopsis (Herve et al., 1996, 1999; Barre et al., 2002), Poplus nigera (Nishiguchi et. al., 2002) and Medicago truncatula (Navarro-Gochcoa et al., 2003). In rice genome (O. sativa, indica) 103 putative LRKs were annotated (Shiu et al., 2004). We have obtained 99 non-redundant amino acid sequences (Shiu, personal communication) and analyzed them using a combinatorial approach: multiple sequence alignment using program ClustalW (Thompson et al., 1994) and prediction mode of BLASTP program (NCBI). Some putative LRKs could not be aligned using ClustalW alone, due to either significant differences in lengths (from 171 to 1311 aa), the positioning of α and β chains or absence of one of the chains in the legume lectin domain. We found 35 putative LRKs possessed only Ser/Thr kinase domains, 4 of them two kinase domains, and no legume-like domains were determined. The remaining 64 LRKs comprised at least one legume-like domain (α, β, or both chains), Ser/Thr kinase domain, and in some case other domains (such as Chase or DUF26 domains). The length of the proteins varied from 349 aa to 1055 aa. The homology among LRKs ranged from 21% to 75%. The identical sequences of the OsLRK1 and the OsLRK2 from O. sativa, japonica had their counterparts in indica genome. The closest homolog of the Oslrk1, the Oslrk2, shared 75% identity on the protein level and 85% similarity at the cDNA level, and was located in the same BAC clone as the Oslrk1, 1 kb downstream of designated polyA signal of the Oslrk1. Another homolog, the Oslrk3, had 59% sequence similarity with and 70% on cDNA level and it was located on chromosome 6, PAC P0019A05.

We carried out Southern-blot hybridization analysis in order to select an Oslrk1 gene specific probe. The genomic DNA isolated from the leaves of WT plants was digested with appropriate enzymes, blotted, and hybridized with probes corresponding to either lectin-domain or 3′UTR of the Oslrk1 gene. When lectin domain was used as a probe, several bands were obtained at low stringent conditions with the exposure time of 2 to 3 hours (data not shown). When the blot was exposed overnight intense smears appeared. On the other hand, when a probe corresponding to 3′ UTR of the Oslrk1 gene was utilized, single band was detected in Southern-blot under similar hybridization and detection conditions (data not shown). Since the Oslrk1 gene specific band was obtained in Southern-blot hybridization with 3′UTR probe, this probe was used to analyze gene expression pattern of the Oslrk1 in Northern-blot hybridizations.

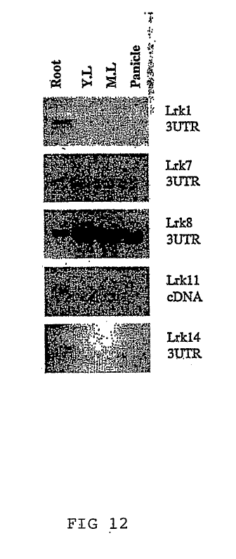

We have analyzed 15 select Oslrks, (including the Oslrk1, analyzed and described in detail herein). Specifically, the Oslrks' position in the rice genome, PAC and BAC clone information, gene sizes and other characteristics were analyzed, with the details provided in Table 2. Furthermore, FIG. 12 shows the mRNA expression results by northern blot using different Irks as probe. In the tissues tested (root, young and mature leaves, and panicles), only Oslrk1, 7, 8, 11, and 14 are expressed. The expression pattern is as seen in the figure.

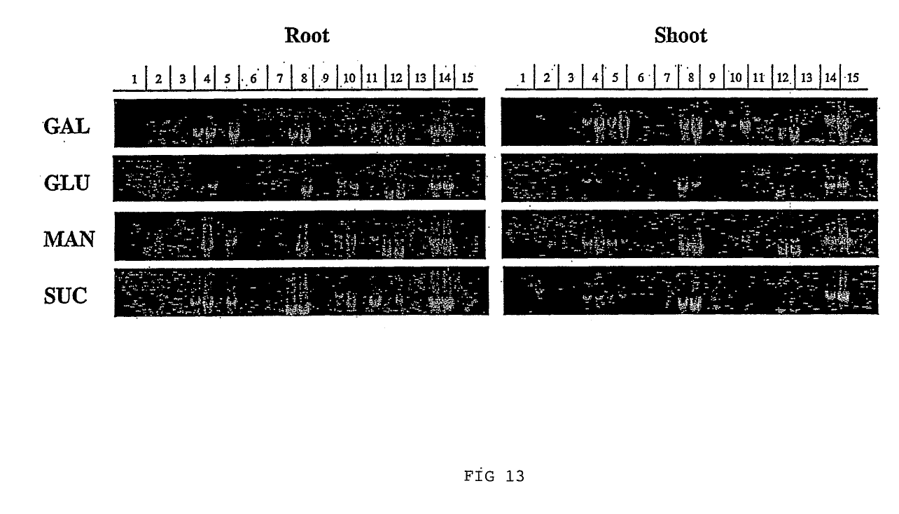

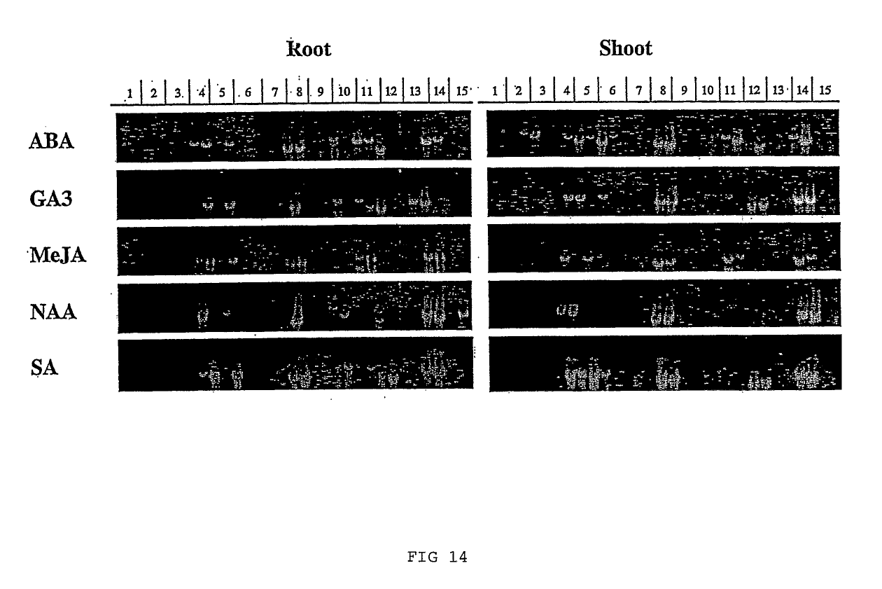

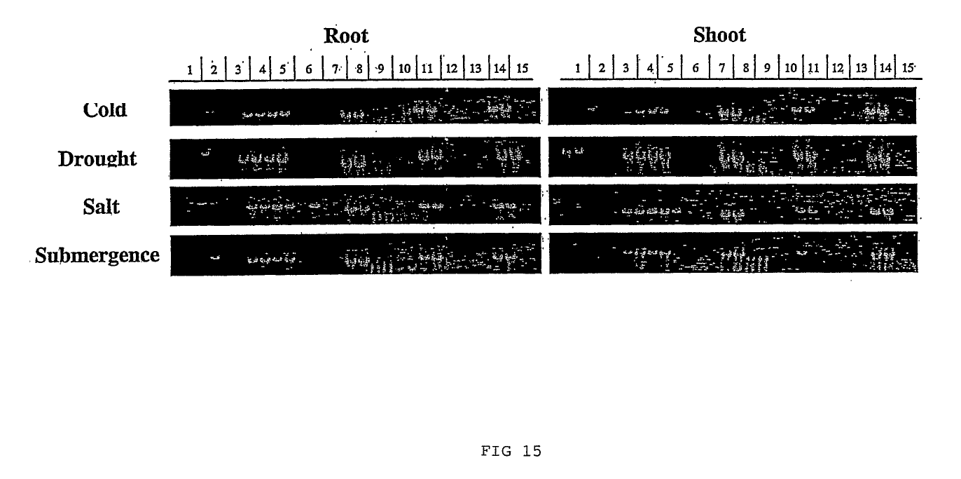

RT-PCRs were carried out on RNA samples isolated from 2 week-old seedlings treated with various sugar (FIG. 13), hormone (FIG. 14), and abiotic stresses (FIG. 15) using primers designated to either 3′ or 5′ Untranslated Regions (UTRs) of the 15 Oslrks found in Table 2. For each Oslrk two lanes in the gel are found, the first lane is the control and the second one treatment samples. These results are also summarized in Table 3.

Table 3 provides a summary of all the above referenced data on the Oslrks, showing the RT-PCR results of various treatments, including sugar, hormone and abiotic stress and northern blot results on various tissues of rice plant grown in normal conditions, and the ESTs, or cDNAs availability in the database. In the table, “I” represents induced expression and “R” represents repressed expression.

TABLE 2
Predicted lectin-receptor kinase family members in annotated rice genome.

| Oslrk | Chr. | | ORF in | Location | Gene length | N° | cDNA length | cDNA/EST |
|---|---|---|---|---|---|---|---|---|
| N° | N° | BAC/PAC | BAC/PAC | (cM) | (bp) | introns | (bp) | (Accession N₀) |
| 1 | 2 | OJ1038_A06 | 136676 | 125.6 | 3265 | 1 | 2211 | AY663848 |
| 2 | 2 | OJ1038_A06 | 132251 | 125.6 | 2752 | 1 | 2205 | AK107391 |
| 3 | 6 | P0019A05 | 75385 | 65.2 | 2375 | 1 | 2289 | AK121604 |
| 4 | 1 | P0010B10 | 56334 | 134.7 | 2091 | no | 2091 | NA |
| 5 | 3 | OSJNBb0081B07 | 91055 | 154.8 | 2105 | no | 2106 | BE040631 |
| 6 | 3 | OSJNBb0094O03 | 29760 | 144.5 | 2033 | 1 | 1926 | CA765300 |
| 7 | 3 | OSJNBb0094O03 | 36553 | 144.5 | 2034 | 2 | 1767 | NA |
| 8 | 7 | OJ1019_E02 | 74985 | 80.8 | 3768 | 5 | 2049 | AK108637 |
| 9 | 7 | OJ1019_502 | 66599 | 80.8 | 4166 | 6 | 2661 | NA |
| 10 | 8 | P0711H09 | 117579 | 103.2 | 2930 | 2 | 2220 | NA |
| 11 | 8 | P0711H09 | 122350 | 103.1 | 3541 | 1 | 2202 | AK109868 |
| 12 | 4 | OSJNBa0081C01 | 85249 | 81.7 | 2001 | no | 2022 | NA |
| 13 | 4 | OSJNBa0081C01 | 86790 | 81.7 | 8482 | 8 | 2040 | NA |
| 14 | 4 | OSJNBa0088A01 | 156052 | 96.0 | 2034 | no | 2034 | NA |
| 15 | 12 | OSJNBb0036A19 | 2352 | 91.9 | 2061 | no | 2061 | NA |

TABLE 3
Summary of RT-PCR, Northern and database analyses on Oslrks.

| | Stress | | | | | | | | Hormones and SA | | | | | | | | | |
|---|---|---|---|---|---|---|---|---|---|---|---|---|---|---|---|---|---|---|
| | Salt | | Cold | | Drought | | Submergency | | IAA | | MeJA | | GA3 | | ABA | | SA | |
| LRKs | Rt | Sh | Rt | Sh | Rt | Sh | Rt | Sh | Rt | Sh | Rt | Sh | Rt | Sh | Rt | Sh | Rt | Sh |
| 1 | I | | | | | I | | | | | | | | | | R | | |
| 2 | R | | I | I | I | R | I | | | | | | | | I | I | | |
| 3 | | | | | | | | | | | | | | | | | | |
| 4 | | | | I | | | | | I | | I | R | I | | I | I | I | |
| 5 | | | | | | | | | I | | I | R | I | | I | I | I | |
| 6 | I | | | | | | | | | | | | | | | R | | |
| 7 | | | | | | | | | | | | | | | | | | |
| 8 | | | | | | | | I | I | | | | I | | | I | I | |
| 9 | | | | | | | | | | | | | | | | | | |
| 10 | | | | | | | | | I | | | | | | R | | I | |
| 11 | | | | R | | | | R | | | | | I | R | | I | I | |
| 12 | | | | | | | | | | | | | | | | I | | |
| 13 | | | | | | | | | | | | | | | | | | |
| 14 | | | | | | | | | | | | | | | | I | | |
| 15 | | | | | | | | | I | | | | | | | | | |

| | Sugars | | | | | | | | | |
|---|---|---|---|---|---|---|---|---|---|---|
| | GAL | | MAN | | SUC | | GLU | | | |
| LRKs | Rt | Sh | Rt | Sh | Rt | Sh | Rt | Sh | Northern | cDNA/EST |
| 1 | | | | | | | | | Rt | |
| 2 | I | R | | | I | R | | | | AK107391 |
| 3 | | | | | | | | | | AK121604 |
| 4 | | I | I | | | | I | R | | |
| 5 | I | I | I | R | I | I | | | | BE040631 |
| 6 | | | | | | | | | | CA765300 |
| 7 | | | | | | | | | | NM_184891 |
| 8 | | I | I | | | | I | R | Rt, Sh, P | AK108637 |
| 9 | | I | | | | | | | | |
| 10 | | I | | | | | | | | |
| 11 | I | R | I | | I | | | | Rt | |
| 12 | | | | | | | | | | |
| 13 | | | | | | | | | | |
| 14 | | I | | | | | | | Rt | |
| 15 | | | | | | | | | | XM_483328.1 |

IAA—indole acetic acid;
MeJA—methyl jasmonate;
GA3—gibberlic acid;
ABA—absisic acid;
SA—salycylic acid;
GAL—galactose;
MAN—mannose;
SUC—sucrose;
GLU—glucose

### EXAMPLE 7

OsLRK1 Domain Organization and its Homologous Sequences in Other Species. The predicted OsLRK1 protein comprises two distinct domains similar to the domains found in other lectin-receptor kinases (Herve C., et al., 1996, 1999; Nishiguch M., et al, 2002; He X.-J. et al, 2004, Navarro-Gochicoa M.-T. et al, 2003): an N-terminal legume-like lectin-receptor domain and C-terminal signal transduction serine/threonine kinase domain (FIG. 4). At the positions N²⁵⁶D²⁵⁷T²⁵⁸, a glycosylation site (consensus sequence Asn-X-Ser/Thr) (FIG. 4) was located (Rudiger H., and Gabius H. J., 2001). Eleven predicted glycosylation sites were observed in the lectin domain. Four residues, E¹⁴⁷, -D¹⁴⁹, -D¹⁵⁶, and -H¹⁶¹ are identical to Mn²⁺-binding amino acids, two of which, D¹⁴⁹ and D¹⁵⁶, also bind Ca²⁺. These cations are required for maintaining the tertiary structure of lectin in such a way that the monosaccharide-binding site is exposed. A signal peptide (20 amino acids in length) and a membrane-spanning domain (23 amino acids in length) were located in the N-terminus of the lectin domain, and in between the lectin and kinase domains, respectively. The serine/threonine kinase domain is 282 amino acids long and is composed of 12 typical protein kinase sub-domains as shown in FIG. 4.

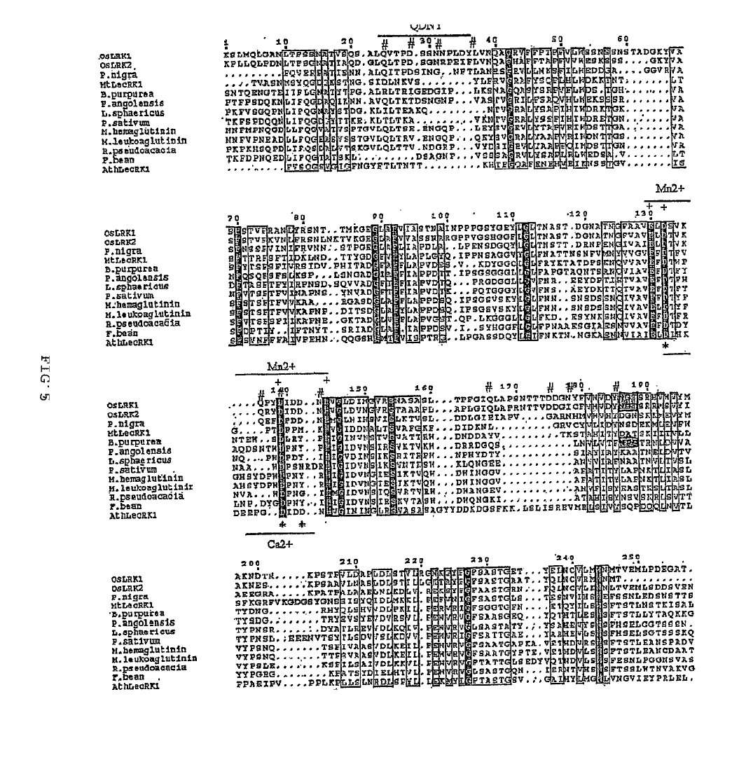

Amino acid sequence of legume-like lectin domain of OsLRK1 revealed strong homology with lectin domains of other lectin-like protein kinases and legume lectins from different species (FIG. 5). For the amino-acid sequence alignment (Notredame, et al., 2000) non-putative and well-characterized (either with solved 3D structure or established function/role) protein sequences were selected. Initially we performed an alignment based on the original lengths of lectin or lectin domains in corresponding LRKs, and then the length of selected sequences were optimized for their better alignment. Among these proteins, the closest homolog of the OsLRK1 lectin-like domain was lectin in LRK (of Populus nigra (PnLRK), which had 51% identity, and 68% similarity and these were the highest scores among the other lectins. Arabidopsis thaliana lectin domain of lectin receptor protein kinase shared 37% identity and 55% homology to the OsLRK1 lectin domain, whereas lectin domain of lectin-like protein kinase from Medicago tranculata shared only 27% identity and 39% homology. The amino acid sequences of legume lectins had a low identity ranging from 24% to 27% and a homology of around 40% to the OsLRK1. Aspartic acid residue (D⁸⁸, FIG. 5 or D¹³⁵, FIG. 4) in the deduced sequence of lectins in lectin-like protein kinases (except MtLECRK1) was replaced either by glutamic residue (as in the OsLRK1, 2 and PnLRK) or by histidine residues (as in AthlecRK1). This residue was shown to be essential in binding lectins to carbohydrates and its substitution resulted in loss of hemagglutination activity in bark lectin from Robinia pseudoacacia (RBL) (Nishiguchi et. al., 1997).

### EXAMPLE 8

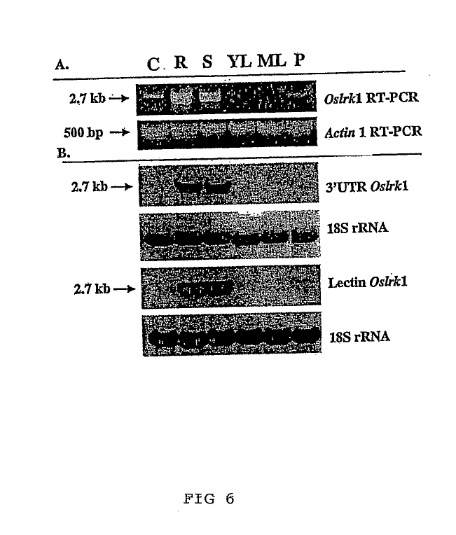

Oslrk1 Gene is Predominantly Expressed in Roots. To determine the expression pattern of the Oslrk1 gene, several approaches have been used, Initially, two sets of experiments were carried out: RT-PCRs using total RNA extracted from callus, 14 days whole seedlings and its roots, young and mature leaves (FIG. 6A) as well as PCRs using cDNA libraries constructed from roots, seedlings and panicles as templates (Materials and Methods). The RT-PCR results showed that the gene was expressed predominantly in roots of the seedlings; however, lower expression level of the Oslrk1 transcript was also observed in panicles and callus (FIG. 6A). In PCRs using cDNA libraries, amplification products were detected in root, seedlings and panicles libraries (data not shown); these results were in accordance with RT-PCR data. In addition to the cDNA library PCRs, Northern-blot analysis was performed with the samples of total RNA used for RT-PCRs. The probes corresponded to the 3′UTR and lectin domain of the Oslrk1 were used for hybridizations. Northern-blots probed with 3′ UTR showed strong expression of Oslrk1 gene in the roots and seedlings, lower expression in panicles and only traces in callus confirming the RT-PCR results (FIG. 6B). When lectin-domain probe was used, the Oslrk1 transcript was detected in the same organs as it was detected with 3′UTR probe in Northern analysis (FIG. 6B).

### EXAMPLE 9

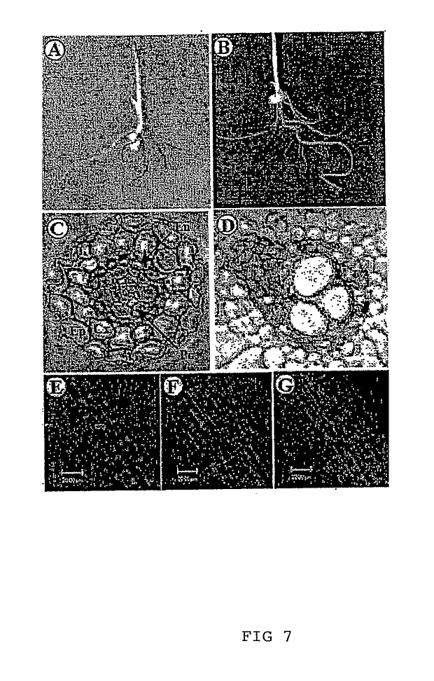

The Oslrk1 Gene is Expressed in Vasculature and Pericycle Cells of Roots. In order to determine the tissue specificity of the Oslrk1 gene expression two constructs were designed: promoter of the Oslrk1 gene fused with either gus or eyfp gene (Materials and Methods). These constructs were introduced in rice and transgenic lines were generated. The T0 and T1 transgenic plants were subjected to histochemical staining and fluorescence studies. Upon 20 minutes of the incubation of four day-old transgenic seedlings in GUS-staining solution, the GUS expression was observed in distal elongation zone of adventitious roots (FIG. 7A). After 1 to 2 hours of incubation the proximal elongation zones and the mature zones of the roots were also stained (data not shown). Seven day-old seedlings showed GUS staining in the elongation and mature zones of adventitious and lateral roots (FIG. 7B). In mature zone of the roots, the vasculature was intensively stained. However, no expression was detected in the root cap. In order to check the cell types of the root that express the Oslrk1 promoter::gus fusion transcript, cross-section analysis of GUS stained roots was carried out. The vascular bundles and pericycle cells of the roots, in particular, showed GUS staining, suggesting the endogenous expression of the Oslrk1 gene in these cells (FIGS. 7C and D). Similar expression pattern was observed when roots of the transgenic plants harboring the Oslrk1 promoter::eyfp gene construct (FIGS. 7E and G). When these transgenic plants were subjected to confocal microscopy, EYFP fluorescence was observed in the vasculature (a central part) of the roots (FIGS. 7B and G). The similar pattern of EYFP fluorescence was detected in the roots at the mature stage. Taken together the GUS expression and EYFP fluorescence data, there was an evidence that the Oslrk1 gene was predominantly expressed in vasculature and pericycle cells of rice adventitious and lateral roots, however, a very low expression in the cortex and epidermal cells can not be excluded.

### Physiological Studies of Oslrk1 Gene

### EXAMPLE 10

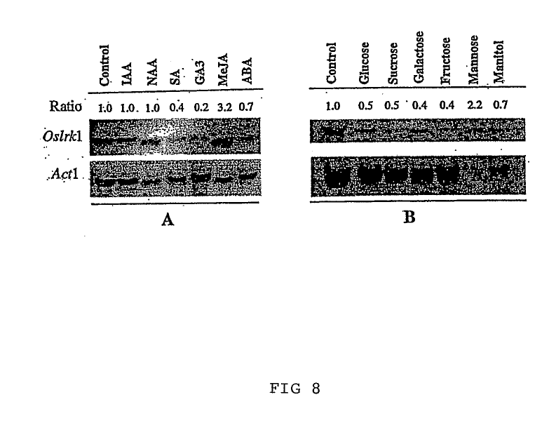

Differential Regulation of Oslrk1 Gene Expression by Hormones. Receptor-like kinases were postulated to be involved in signal transduction events as they perceive a signal via a ligand-binding extracellular domain and transmit it through the serine/threonine kinase domain into the cytosol (Shiu and Bleecker, 2001). In order to know possible factors influencing the Oslrk1 gene regulation, we analyzed promoter of the Oslrk1 (1.5 kb) for the presence of cis-acting elements using the program PlantCARE (Lescot et al., 2002). Three TGAGC motifs observed in MeJA inducible genes were found in promoter of the Oslrk1 at the positions −186, −657 and −1190. Apart from these, three GA₃ inducible motifs (P-box-CCTTxt) −101, −206 and −740 bp upstream of the Oslrk1 ORF were located. In addition, five salicylic acid inducible motifs (TCA-elementcAGAAa/ga) at the positions of −321, −601, −604, −855 and −1135 were detected. The presence of MeJA, GA and SA responsive cis-elements in the Oslrk1 promoter indicated possible regulatory roles of these factors. The promoter analysis led us to study the effect of different hormones and compounds on the Oslrk1 transcription. The WT seeds were germinated for seven days then seedlings were transferred to the medium containing one of the following chemicals: NAA, MeJA, GA₃ or ABA at concentration 100 uM and SA (5 mM) for 24 hours (Materials and Methods). The total RNA isolated from the untreated and treated seedlings were used in Northern-blot hybridization using 3′ UTR of the Oslrk1 gene as a probe. The treatment with GA₃ and SA repressed the Oslrk1 gene expression (FIG. 8A) by 2.5 and 3.3 times respectively. On the other hand, MeJA induced the Oslrk1 transcript; the intensity of the hybridization signal was increased by 3.2 times when compared to the control, whereas no significant effect on transcription was observed when seedlings were treated with IAA and ABA. In order to confirm the level of induction of the Oslrk1 gene by MeJA over time, in another series of experiments, a time course was performed after hormone treatment. Seven day old seedlings were sprayed with 100 uM of MeJA solution and they were harvested in 1, 2, 4, 6, 8, and 12 hours. The Oslrk1 expression was gradually induced over time with a maximum at 8 hours with approximately 3 times that of untreated sample and stabilized up to 12 hrs after treatment (data not shown).

### EXAMPLE 11

Oslrk1 Gene Transcript Showed Differential Expression in Response to Sugars. Lectins are known as carbohydrate-binding proteins that specifically recognize diverse sugar structures (Vijayan and Chandra, 1999). Since the OsLRK1 possessed a legume-like lectin domain we were interested to test the response of the Oslrk1 expression to different sugars. Five different sugars: glucose (Glu), sucrose (Suc), fructose (Fm), Gal and Man were used for this study along with Mannitol as an osmotic control (Methods). Northern-blot hybridizations were carried out on the total RNA isolated from treated seedlings. FIG. 8C shows the Oslrk1 expression in response to these monosaccharides and Suc in which, all tested sugars except Man repressed the gene expression. The transcript level of Oslrk1 was not altered in response Mannitol indicating that this gene is not osmotically regulated.

### EXAMPLE 12

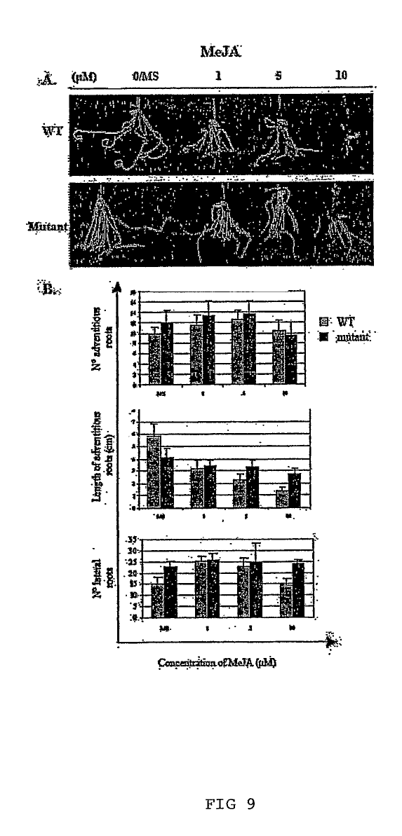

The Oslrk1 Mutant Showed Altered Response to Phytohormones. In order to study the effect of phytohormones on the Oslrk1 mutant, seeds were grown in medium containing different concentrations of either MeJA (1, 5, and 1011 m) (FIG. 9A) or GA3 (0.5, 1, 5 and 10 μm) (data not shown) along with WT as a control The number and length of adventitious roots, number of lateral roots and shoot length were measured. Upon MeJA treatment, the shoot length was decreased in both WT and mutant but there were no significant differences between them with increasing concentration. However, the number of adventitious roots gradually increased up to 5 uM in the WT seedlings and the mutant, whereas at 10 uM the effect of MeJA was opposite to the effect observed at 5 uM (FIG. 9B). In contrast, the length of adventitious roots in WT gradually decreased upon increased concentration of MeJA (up to 10 uM) with approximately 75% reduction in length at 10 uM, however, the length of adventitious roots of the Oslrk1 mutant was not reduced significantly. In the WT seedlings the number of the lateral roots was increased at 5 uM and decreased by at 10 uM. In contrast, in mutant there were no significant changes in the number of lateral roots were observed FIGS. 9A and B). These results suggested that the Oslrk1 mutant was less sensitive to MeJA application.

On the other hand, when seedlings were grown for two weeks in GA3 containing MS agar, no significant differences were observed in the behavior of mutant compared to that of WT, except, the length of adventitious roots. The adventitious root length of WT increased by 50% at 10 uM concentration of GA3, but it was not altered in the mutant (data not shown).

### EXAMPLE 13

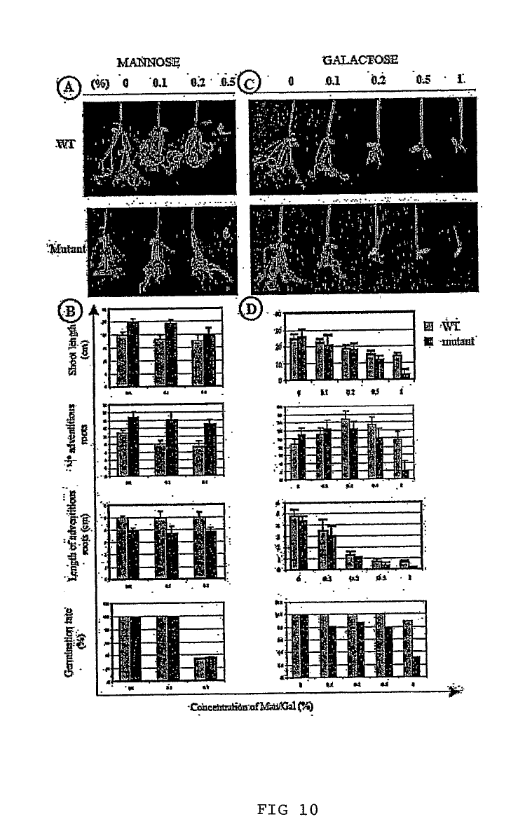

Oslrk1 Mutant Demonstrated Hypersensitivity to Galactose. The altered expression of the Oslrk1 by different sugars described above led us to study the response of the mutant to these monosaccharides and sucrose. The WT and the mutant seeds were germinated and grown in Gal, Man, Glu, Fm along with Suc at different concentrations (ranging from 0.1% to 1%) and Suc alone containing medium as a control. The following parameters were taken into account during observation: the shoot length, the number and the length of adventitious roots and the germination rate. No significant differences were revealed in the behavior of the mutant and the WT upon the treatment of seedlings with various concentrations of Glu, Fm and Man, but a differential response was observed when seeds were germinated and grown in Gal containing medium. Out of these three sugars tested, Man was selected as an example as its inhibitory effect was similar in seeds and seedlings of WT and mutant as shown in FIGS. 10A and B. The seed germination was severely affected by Man. At concentration of mannose 0.5%, seeds of both WT and mutant plants did not germinate. No obvious effect on any parameters tested was noticed at concentration of mannose 0.1%, but at concentration 0.2%, the seed germination rate dropped to 35% in both the WT and the mutant.

On the other hand, the WT and mutant seedlings showed statistically significant differential response when grown on Gal-containing medium (FIGS. 10C and D). The adventitious root number and, especially, the length were strongly inhibited by Gal in mutant compared to the WT (FIGS. 10B and C). At 1.0% of the Gal, the shoot length decreased by half in the mutant, whereas only by 15% in the WT seedlings. The number of the adventitious roots was more or less same in the WT but in mutant they were drastically reduced by 85%. In addition to this, the length of adventitious roots in the WT and mutant were reduced at 1.0% Gal with severe reduction in mutant. Apart from this, the germination rate of the WT seeds dropped to 85% whereas in the mutant it dropped to 30%. Based on the results obtained, we concluded that the Oslrk1 mutant exhibited hypersensitivity to Gal.

### Methods

Genomic DNA Isolation and Southern-blot Hybridization. Five μg of the genomic DNA was isolated from the leaves (Dellaporta et al., 1983), digested with the appropriate restriction enzymes (EcoRI, PstI, PvuII), fractionated on 0.8% agarose gel and transferred onto a Hybond-N⁺ membrane (Amersham Biosciences, Little Chalfont, Bucks, UK). DNA blots were hybridized with dogoxigenin (DIG)-labeled probes in DIG easy Hyb solution (Roche Applied Science, Mannheim, Germany) at 42° C. After hybridization, membranes were washed twice with 2×SSC and 0.1% SDS for 15 min, then twice with 0.5×SSC and 0.1% SDS for 15 min at 68° C. Detection was carried out according to manufacturer using DIG Wash and block Buffer set and chemiluminescent substrate CDP-Star (Roche). DIG labeled probes were synthesized using PCR DIG Probe Synthesis Kit (Roche) with a following primers:

| | |
|---|---|
| GUS probe: | |
| GusF: | 5′-CATTTGAAGCCGATGTACC-3′an |
| GusR: | TATCGGTGTGAGCGTCGCAG-3′; |
| BAR probe: | |
| BarF: | 5′-CATCAGATCTCGGTGACG′-3 |
| and | |
| BarR: | 5′-TCGTCAACCACTACATCG-3′; |
| 3UTR of Oslrk1: | |
| LRPK1F2: | 5′-GTCGCTGTTCG-TACTACG-3′ |
| and | |
| 3UTRR5: | 5′-AGGATCGAACGCAAAGAG-3′; |
| Lectin probe: FRT1 | 5′-CAACCCCCACCCTTTTCTCCA-3′ |
| and | |
| RRT2: | 5′-GGCCCGCCGAAGAGGTG-3′ |

RNA Isolation and Northern-blot Analysis. RNA was isolated from different organs of rice plant at different developmental stages (roots of 10-14 days old seedlings and 2 months old plants, 10-14 days old seedlings, young and mature leaves, panicles at the flowering stage and calli) using a RNeasy Plant mini kit (Qiagen,). The RNA (10 ug) was fractionated on a 1.2% denaturating formaldehyde agarose gel and transferred onto a Hybond-N⁺ membrane (Amersham). The bolts were hybridized with dogoxigenin (DIG)-labeled probes in DIG Easy Hyb solution (Roche) at 50° C. After hybridization, membranes were washed twice with 2×SSC and 0.1% SDS for 15 min, then twice with 0.5×SSC and 0.1% SDS for 15 min at 68° C. Detection was carried out according to manufacturer using DIG Wash and Block Buffer set and chemiluminescent substrate CDP Star™ (Roche). DIG-labelled probe corresponded to 3′UTR and lectin domain of Oslrk1 was used. For the control of RNA quantity, DIG-probe corresponding to rice Actin1 gene was synthesized using the following primers:

| | |
|---|---|
| 414F: | 5′-CTCTCAACCCCAAGGCCAATC-3′ |
| and | |
| 1113R: | 5′-AGGGCAGCGGAAACGCTCAG-3′ |

The RT-PCRs and Northern blots were repeated at least thrice and similar results were obtained. The quantification of the intensity of the hybridization signals was performed using Quantity ONE 4.5.0 software in Gel doc XR documentation system (Bio-Rad Laboratories, Inc., USA). The level of actual expression was calculated as a ratio of intensity of specific signal (Oslrk1 3′UTR probe) to the intensity of signal of positive control (rice Act1).

RT-PCR Analysis.

The Oslrk1 cDNA (GenBank accession number AY663848), 2701 bp in length, was amplified by RT-PCR using total RNA extracted from the roots of 14 day old seedlings with a One step RT-PCR kit (Qiagen). The PCR reaction was carried out on a PTC-100HB-96 thermocycler (MJResearch Inc., Watertown, Mass., USA) under the following conditions: 50° C. for 30 min, 95° C. for 15 min, followed by: 94° C. for 40 sec, 58° C. for 40 sec, 72° C. for 3 min, for 35 cycles, using the following primers: 5UTR-F7: 5′-TTTCGTGGCCAACTCCT-3′, 5UTR-F6: 5′-CCCCTTGCTACATCACC-3′ and 3UTR-R5: 5′-AGGATCGAACGCAAAGAG-3′. For the expression study, RT-PCR was performed with total RNA used in Northern-hybridization. For the internal control, the primers corresponding to the rice Actin1 (414F and 113R) described above were used. For PCRs using cDNa libraries, primers specific for 3′ UTR of the Oslrk1 gene were utilized to obtain amplification product 500 bp in length.

### Physiological Studies on the Oslrk1 Mutant

Plant Growth Conditions. Surface sterilized rice seeds were germinated and grown in cell culture containers (Phytatray II Sigma cell culture, USA) on 0.8% agar MS medium (Murashige and Skoog medium, Sigma., St. Louis Mo., USA) containing 1% of Suc for 2 weeks under 16/8 hours photoperiod. All chemicals used for the various treatments were purchased from Sigma.

Treatment of WT Seedlings with Hormones and Sugars. Two week old WT seedlings were transferred to MS medium containing 100 μM of different hormones, such as Indole 3-Acetic Acid (IAA), Abscisic acid (ABA), Methyl Jasmonate (MeJA), or Gibbrellic Acid (GA3) and 5 mM of Salicylic Acid (SA), and were grown for 24 hours under normal day/light conditions. Total RNA was extracted from root of the seedlings and whole seedlings.

Time course expression studies of the MeJA effect were carried out with two week old seedlings after spraying with 100 μM of MeJA and total RNA from seedlings harvested at different time intervals (Oh, 1 h, 2 hrs 4 hrs, 6 hrs, 8 hrs and 12 hrs) was purified.

To study the effect of different sugars on the Oslrk1 transcript, surface sterilized WT seeds were germinated on MS medium containing 0.8% agar without sucrose, under dark conditions for 1 week. Then, seedlings were transferred to MS medium containing 175 mM of different sugars including sucrose, D(+)glucose, P-D(−)fructose, D(+/−, mixture of isomers)mannose, D(+)galactose and incubated 48 hours in normal day/light conditions.

Germination of WT and mutant seeds in galactose/mannose containing media. WT and mutant seeds were germinated separately on MS medium containing 0.8% agar and 1% sucrose. In addition, D(+)galactose/D(+/−)mannose was added in different concentrations (0.1, 0.2, 0.5, and 1.0%) along with a control (MS with 1% of sucrose). At 14 days after germination, the observations for adventitious root number and length (cm), shoot length (cm) and germination rate (%) were recorded.

Growth of WT and Mutant Seedlings in MeJA Containing Medium WT and mutant seeds were germinated and grown in MS medium (1% Suc) containing different concentrations of MeJA (1 μM, 5 μM, and 10 μM) for two weeks. No MeJA was added in the control. The number and length (cm) of adventitious roots as well as the number of lateral roots were measured.

Promoter Oslrk1::gusA and Promoter Oslrk1::eyfp Constructs. Two constructs containing promoter of Oslrk1 (1.5 Kb) fused with either gusA or eyfp genes were generated. A 1.5 kb length of upstream region was selected based first on the finding of stop codon of another annotated gene approximately 2 kb upstream of ATG of the Oslrk1. Secondly, we performed a search for the cis-regulatory elements on different regions upstream ATG codon of the Oslrk1 using the PlantCARE program (Lescot, et al., 2002) and database for the search of cis-regulatory elements and found that the same set of elements were present along the 1.5 kb and 2 kb sequence lengths, but the number of elements varied. Since the 1.5 kb region comprised all regulatory elements, at least those available in the database, we amplified this region and used it for promoter-reporter gene fusion constructs.

A fragment (1 kb) corresponding to the Oslrk1 promoter was amplified in PCR using genomic DNA (100 ng) extracted and purified from the leaves of WT seedlings and the following primers: PromOslrk1F (CATTGCTTGGTCATTGG) and PromOslrk1R (AGGTCTGCGAGGAGTT) corresponding to promoter sequence of the Oslrk1 gene. The PCR was carried out in 50 μl containing 1×PCR buffer, 1.5 mM MgCl, 200 μM of each dNTP and 1.5 U of HotStar Taq-polymerase (5 U/μl) (Qiagen,). Cycling program included: 95° C. for 15 min, 94° C. for 40 sec., 58° C. for 40 sec., 72° C. for 1 min, 30 cycles. The amplified fragment was purified from the 1% agarose gel and sub-cloned into pGEM-T Easy vector using pGEM-T Easy Vector System I (Promega, Madison, Wis., USA, Cat. NA1360). Promoter Oslrk1 in pGEM-T Easy vector was cleaved with NcoI and SalI restriction enzymes, then, fragment of 1.4 kb corresponding to the Oslrk1 promoter was purified from the gel and used directly for cloning in pCAMBIA 1301 vector (CAMBIA), digested with the same restriction enzymes (promoter Oslrk1::gusA). The ligation was performed using Rapid DNA Ligation Kit (Roche) for overnight at 4° C.

PromOslrk1::gusA construct in pCAMBIA 1301 was digested with XbaI and NcoI restriction enzymes, fragment corresponding to 1.4 kb was cloned in the eyfp containing construct pSSZ32 (Kolesnik et. al., 2004) cleaved with the same restriction enzymes (Oslrk1 promoter::eyfp).

Both constructs were transformed in rice calli by Agrobacterium-mediated transformation (Hiei et al., 1994); transgenic calli were selected and regenerated.

GUS Staining Analysis and Confocal Microscopy. For the GUS expression study, different organs of rice plant at different stages of development were collected into GUS staining solution (Kolesnik et al., 2004) and incubated at 37 C. Then staining solution was replaced with 70% ethanol to remove chlorophyll content, followed by microscopic visualization of stained tissues under a LEICA MZ12 microscope (Lieca Microsystems, NuBloch GmbH, Heidelberger). For anatomical details, the stained roots were infiltrated, embedded, and mounted using the LEICA HISTORESIN Embedding Kit, (Leica Microsystems), according to the manufacturer's protocol. The mounted samples were sectioned using a triangular glass knife (clearance angle 4°-7°), on a motorized microtome with retraction, LEICA RM 2165 (Leica Microsystems), and observed under a Nikon Eclipse 80i microscope.

EYFP expression in transgenic plants harboring Promoter Oslrk1::eYFP construct was analyzed using LSM Ziess Confocal microscope (Model 510 META) using 488 nm wavelength. The imaging was performed with Zeiss LSM Image Browser version 3, 2,0,70.

Statistical Analysis

The parameters listed in Table 2, were subjected to t-test and the probability values (pValues) were calculated. The behavior of WT and mutant characteristics in response to increasing concentrations of MeJA (FIG. 9) and Mannose and Galactose (FIG. 10) were tested statistically by two-way Analysis of Variance (ANOVA). This test was used to investigate the difference in the change of patterns for both treatments (Mutant and wild type), in which we analyzed and calculated the pValues for the interaction effects. Also we have performed General Linear Model ANOVA to investigate the type of pattern changes for both treatments.

### Discussion

Several reports have shown that the lectin-like receptor kinases were encoded by multigene families. In Arabidopsis thaliana 10 lectin-like kinases were described (Herve et al., 1999) and 42 genes were predicted (Barre et al., 2002; Navarro-Gochicoa et al., 2003); in Medicago truncatula at least 9 LRKs were reported (Navarro-Gochicoa et al., 2003); and in Populus nigra the presence of several LRK homologs in genome was demonstrated by Southern hybridizations (Nishiguchi et al, 2002). We have also shown by ClustalW and BLASTNP homology searches in NCBI databases that the Oslrk1 gene belongs to a multi-gene family comprising of al least 64 members. They were distributed on different chromosomes forming clusters of two to three genes. The events of clustering of RLKs were described in rice where more than 42% of genes found in tandem repeats (Shiu et al., 2004). The similar tandem organization of RLKs was published in Arabidopsis (34%; Shiu et al., 2001), but in rice, particularly, the degree of RLK tandem duplication higher than in Arabidopsis. Rice LRKs shared homology between 23 to 75% on the protein level.

We have identified a mutant line with the Ds insertion in the closest homolog of the Oslrk1, the Oslrk2. The detailed observation of the Oslrk2 mutant phenotype did not reveal any changes in characteristics of roots and aerial part compared to the WT, even though it was a null mutant as the insertion landed in the 1^{st} exon of the gene. We could not demonstrate the expression of the Oslrk2, although the corresponding cDNA from immature seeds was found in KOME database with a 99% match in the sequence. The seed yield was thoroughly analyzed in the homozygous mutant, but no differences were observed compared to the WT (data not shown). The lack of the obvious phenotype in the Oslrk 2 mutant might suggest that this gene is functionally redundant under the normal conditions.

The lectin like-receptor kinases belong to a class of receptor kinases with N-terminal ligand binding lectin-like extracellular domain, transmembrane spanning domain and cytosolic kinase domain. This class of receptor kinases was reported in Arabidopsis thaliana, Medicago truncatula and Populus nigra (Herve et al., 1996, 1999; Nishiguchi et al., 2002; Navarro-Gochicoa et al., 2003). In RLKs extracellular domain is determined by the specificity of the recognized ligand. The OsLRK1 possessed a legume-like lectin as a recognition domain, as shown in other plants from Leguminosea (Van Damme et al., 1998). Legume lectins are unique group of lectins as they contain divalent cations binding stites. The OsLRK1 has conserved residues responsible for Mn²⁺ and Ca²⁺ binding in its lectin domain (FIG. 5). These cations are essential for the carbohydrate-binding activity of lectins (Sharon and Lis, 2002).

The aspartic amino acid involved in monosaccharide binding was found in all legume lectins. This residue was replaced in the OsLRK1 by glutamic acid and it corresponded to Glu¹³⁵ in the complete amino acid sequence (FIG. 4) and in Glu⁸⁸ of the aligned amino acid sequence (FIG. 5). Similar replacement was observed in all lectin-like kinases, except Medicago truncatula lectin kinase (MtLecRK1;1), by either glutamic acid (OsLRK1, PnLRK) or histidine (AthLecRK1) residues. Other conserved residues involved in sugar recognition corresponded to Gly¹¹², Asn¹²⁴, Gly²²⁷, Ala²³⁰ in deduced sequence of lectin domain were also found in the OsLRK1 protein. Besides the lectin domain, the OsLRK1 also possessed a well-conserved serine/threonine kinase domain at the C-terminus of the OsLRK1. The lectin-receptor kinase of Lombardi poplar (PnLRK; Nishiguchi et al., 2002) and Arabidopsis (AtLecRK2; He et. al., 2004) were shown to be capable to autophosphorylation at the conserved serine/threonine residues in the kinase domain. Similar phosphorylation motifs (DIKPS and GTLGYIAPE) were observed in the kinase domain of the OsLRK1.

Expression analysis of the Oslrk1 by RT-PCR, cDNA libraries screening and Northern-blots demonstrated that the Oslrk1 was predominantly expressed in roots, similar to MtLec1;1, MtLec7;2 and MtLec7;3 from Medicago truncatula (Navarro-Gochicoa et al., 2003), PnLRK1 from Lombardi poplar (Nishiguchi et al., 2002) and AtlecRK2 from Arabidopsis (He et al., 2004). We have demonstrated by RT-PCR and Northern-blot-hybridization that the Oslrk1 was predominantly expressed in roots of seedlings and mature plants. The BLASTN homology search in NCBI EST databases revealed the EST sequences derived from rice roots of one-week-old seedlings (Accession N BE039888) and from roots of bread wheat (Accession NCD87254) identical to 5′ of the Oslrk1 providing the additional evidences of the expression of this gene in roots. Additionally, the analysis of the transgenic plants harboring the Oslrk1 promoter::gusA or eyfp confirmed that the Oslrk1 predominantly expressed in the vasculature of the roots. The expression of the OsLRK1 in vasculature might indicate either this gene is involved in vasculature development or in carbon or nutrient transport from source organs to the roots. Cross-sections of roots of WT and mutant did not reveal differences in organization of vascular tissues, which suggests that the OsLRK1 hardly plays a role in vasculature development. On the other hand, the mutant plants displayed the expanded root system, which suggested that carbon (nutrient) deposition from the source organs to the root cells might be increased by the knockout of the OsLRK1 resulting in root expansion.

The OsLRK1, a putative trans-membrane protein might perceive a signal from the apoplast and transmit it to the cytosol of the cell. The RLKs were regulated by various biotic and biotic stress factors, potentially involved in signal transduction pathways (McCarthy and Chory, 2000). The Arabidopsis AtlecRK-al and Lombardi polar PnLRK were induced by wounding (Riou et al., 2002; Nishiguchi et al., 2002), whereas AtlecRK2 was induced by salt (He et al., 2004). However, treatment of Populus nigra leaves with different hormones such as ABA and JA and SA had no effect on the expression of the PnLRK gene (Nishiguchi et al., 2002). These reports and the presence of several MeJA, GA3 and SA responsive cis-acting elements in the Oslrk1 promoter led us to study the Oslrk1 gene regulation by different hormones (IAA, GA3, MeJA, and ABA) and SA. The differential Oslrk1 gene regulation was demonstrated in response to hormones and SA. The expression level was elevated in response to MeJA and repressed in response to GA3 and SA.

Despite relatively low levels of MeJA in roots (Berger et al., 1996), in Arabidopsis it was shown that MeJA strongly inhibited root growth (Staswick et al., 1992, Berger et al., 1996, Ueda et al., 1995). Similarly, in rice, development of adventitious roots was also inhibited by MeJA at concentration more than 3 μM (Moons et al., 1997); however, the molecular basis underlying the root inhibition is still not known. The fact that the Oslrk1 transcript was induced by MeJA and the mutant was less sensitive to MeJA treatment suggested that the OsLR1 responds or mediates MeJA signalling in regulation of root growth.

In contrast to MeJA, GA3 suppressed the Oslrk1 transcript. The GA3 is known as root growth promoting factor, it mediates auxin role in promoting root growth trough the inhibition of RGA and GAI transcription. (Fu and Harberd, 2003). The fact that the Oslrk1 transcript was up regulated by MeJA (root growth inhibitor) and inhibited by GA3 (root growth promoter) was correlated with the assumed function of the Oslrk1 as a root growth inhibitor. Additionally, the length of adventitious roots was less altered in the mutant compared to the WT upon GA3 treatment, which might suggest the role of the OsLRK1 in mediating signal from GA3 in regulation of the root length.

A cross talk between hormone and stress signaling in plants is the current issue in understanding the plant response to abiotic stimuli and regulation of developmental processes. In Arabidopsis, salt-induced AtlecRK2 was shown to be up regulated by ethylene (He et al., 2004) and in rice roots salt inducible SalT gene was accumulated in response to JA (Moons et al., 1997). The physiological role of LRKs remains unknown. The assumption that legume-like lectin domains in LRKs might be involved in carbohydrate binding was not confirmed, moreover the substitution of the key amino acids involved in sugar binding in the most of the LRKs led to the speculation that lectin domains hardly can recognize monosaccharides (Herve et al., 1996, 1999). In the OsLRK1 the most of the key residues involved in sugar binding in other legume lectins were conserved except of Asp⁸⁸. In Arabidopsis the activation of Athlecrk-al transcript by oligogalacturonide was reported (Riou et al., 2002). We also have shown that the Oslrk1 transcript was repressed by various sugars except Man. The fact that Mannitol did not increase the transcript level of Oslrk1 suggested that this gene is not osmotically regulated. Despite the differential regulation of Oslrk1 expression by different carbohydrates, the mutant showed altered response only in response to Gal.

In plants, especially, monocots, Gal is an important constituent of galactolipids, cell-wall polysaccharides and glycoproteins (Dormannn et al., 1998). Gal was shown as a very strong inhibitor of root expantion in Arabidopsis (Dormann and Benning, 1998), barley (Farrar et al., 1994), oat (Cheung and Cleland, 1991), tomato (Huges et al., 1974), wheat (Knudson, 1917) maize and other species (Yamamoto et al., 1988). In barley, the inhibitory effect of Gal on root cell expansion was demonstrated to occur due to reduced import of the carbon to the growing organs (Thorpe et al., 1999). The Oslrk1 mutant exhibited hypersensitivity in response to Gal when compared with WT (FIG. 10), which suggests that the OsLRK1 plays a role in Gal signaling to minimize its effect in root growth inhibition.

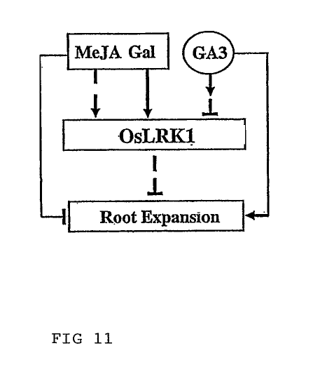

Based on our results on the Oslrk1 expression analysis and physiological studies of the mutant we propose a model for the OsLRK1 in rice as shown in FIG. 11. The Oslrk1 as a negative regulator of root growth as the loss of this gene function results in expanded root system. The GA3 has been shown to promote root growth and on the other hand, MeJA, salt stress and Gal have been shown to inhibit the root growth. Repression of the Oslrk1 gene expression by GA3 suggests that this gene may be involved in root growth inhibition. Induction of the Oslrk1 transcript in WT and reduced sensitivity of the mutant to MeJA, showed that the OsLRK1 perceives signals from MeJA either directly or indirectly and inhibits the root growth. Repression of Oslrk1 transcript by Gal as shown by expression studies and the hypersensitivity of mutant to Gal indicated that the OsLRK1 is involved in response to galactose and inhibit root expansion. In summary, our results we proposed a role of the OsLRK1 as a negative regulator of root expansion and mediator of phytohormones and sugar signaling in regulation of root growth.

### REFERENCES

Barre, A., Herve, C., Lescure, B., and Rouge, P. (2002). Lectin-receptor kinases in plants. Crit. Rev. Plant Sci. 24, 379-399.
Becraft, P. W. (2002). Receptor kinase signalling in plant development. Annu. Rev. Cell Dev. Biol. 18, 163-192.
Berger, S., Bell, E., and Mullet, J. E. (1996). Two methyl jasmonate-insensitive mutants show altered expression of AtVsp in response to methyl jasmonate and wounding. Plant Physiol. 111, 525-31.
Cheung S. P., and Cleland, R. E. (1991). Galactose inhibits auxin-induced growth of Avena coleoptiles by two mechanisms. Plant Cell Physiol. 32, 1015-1019.
Chin, H. G., Choe, M. S., Lee, and S. H. et al. (1999). Molecular analysis of rice plants harboring Ac/Ds transposable element-mediated gene trapping system. Plant J. 19, 615-623.
Dellaporta, S. L., Wood, J., and Hicks, J. B. (1983). A plant DNA minipreparation: version 2. Plant Mol. Biol. Rep. 14, 713-726.
Dormann, P., and Benning, C. (1998). The role of UDP-glucose epimerase in carbohydrate metabolism of Arabidopsis. Plant J. 13, 641-52.
Farrar, J. F., Minchin, P. E. H., and Thorpe, M. R. (1994). Carbon import in barley roots: stimulation by galactose. Journal Exper. Bot. 45, 17-22.
Fu, X., and Harberd, N. P. (2003). Auxin promotes Arabidopsis root growth by modulating gibberellin response. Nature 421, 740-743.
Hardie, G. D., Carling, D., and Carlson, M. (1998). The AMP-activated/SNF1 protein kinase subfamily: Metabolic Sensors of the eukariotic cell? Annu. Rev. Biochem. 67, 821-855.
He, X.-J., Zhang, Z.-G., Yan, D.-Q., and Chen., S.-Y. (2004). A salt responsive receptor-like kinase gene regulated by the ethylene signaling pathway encodes a plasma membrane serine/threonine kinase. Theor. Appl. Genet. April 6 (on line)
Herve, C., Dabos, P., Galaud, J. P., Rouge, P., and Lescure B. (1996) Characterization of an Arabidopsis thaliana gene that defines a new class of putative plant receptor kinases with an extracellular lectin-like domain. J. Mol. Biol. 258, 778-788. Herve, C., Serres, J.,
Dabos, P., Canut, H., Barre, A., Rouge, P., and Lescure B. (1999) Characterization of the Arabidopsis lecRK-α genes: members of a superfamily encoding putative receptors with an extracellular domain homologous to legume lectins. Plant Mol Biol. 39, 671-82.
Hiei, Y., Ohta, S., Komari, T., and Kumashiro, T. (1994). Efficient transformation of rice (Oryza sativa L.) mediated by Agrobacterium and sequence analysis of the boundaries of the T-DNA. Plant J. 6, 271-282.
Jinn, T.-L., Stone, J. M., and Walker, J. C. (2000). HAESA, an Arabidopsis leucinerich repeat receptor kinase, control floral organ abscission. Genes Dev. 14, 104-116. Knudson, L. (1917). The toxisity of galactose and mannose for green plants and the antagonistic action of other sugars towards these. American Journal of Botany 4, 430-437.
Kolesnik, T., Szeverenyi, I., Bachmann, D., Kumar, S., Jiang, S., Ramamoorthy, R., Cai, M., Ma, Z., Sundaresan, V., and Ramachandran, S. Establishing an Efficient Ac/Ds Tagging System in Rice: Large-Scale Analysis of Ds Flanking Sequences (2004). Plant J. 37, 301-314.
Lescot, M., Dehais, P, Thijs, G., Marchal, K., Moreau, Y., Van de Peer., Y., Rouze, P., and Rombauts, S. (2002). PlantCARE, a database of plant cis-regulatory elements and portal to tools for in silico analysis of promoter sequences. Nucleic Acids Res. 30, 325-327.
Liu, Y. G., and Whittier, R. F. (1995). Therminal assimetric interlaced PCR: automatable amplification and sequencing of insert end fragments from P1 and YAC clones for chromosome walking. Genomics 25, 674-681.
Loris, R. (2002). Principles of structures of animal and plant lectins. Biochim. Biophys. Acta 1572, 198-208.
McCarty, D. R., and Chory, J. (2000). Conservation and innovation in plant signalling pathways. Cell 103, 201-209
Moons, A., Prinsen, E., Bauw, G., and Montagu, M. (1997). Antagonistic effects of abscisic acid and jasmonates on salt stress-inducible transcripts in rice roots. Plant Cell 9, 2243-59.
Morris, E. R., and Walker, J. C. (2003). Receptor-like protein kinases: the keys to response. Current Opin. Plant Biol. 6, 339-342.
Navarro-Gochicoa, M. T., Camut, S., Timmers, A. C., Niebel, A., Herve, C., Boutet, E., Bono, J. J, Imberty, A., and Cullimore, J. V. (2003). Characterization of four lectinlike receptor kinases expressed in roots of Medicago truncatula Structure, location, regulation of expression, and potential role in the symbiosis with Sinorhizobium meliloti. Plant Physiol. 133, 893-910.
Nishiguchi, M., Youshida, K., Sumizomo, T., and Tazaki, K. (1997). Studies by site-directed mutagenesis of the carbohydrate-binding properties of bark lectin from Robinia pseudoacacia. FEBS lett. 403, 294-298.
Nishiguchi, M., Youshida, K., Sumizomo, T., and Tazaki, K. (2002). A receptor-like protein kinase with a lectin-like domain from lombardy poplar: gene expression in response to wounding and characterization of phosphorilation activity. Mol. Genet. Genomics 267, 506-514.
Notredame, C., Higgins, D., and Hering a, J. (2000). T-Coffee: A novel method for multiple sequence alignments. Journal Mol. Biol. 302, 205-217.
Ramachandran, S., and Sundaresan, V. (2001). Transposons as tools for functional genomics. Plant Physiol. Biochem. 39, 243-252.
Riou, C., Herve, C., Pacquit, V., Dabos, P., and Lescure, B. (2002) Expression of an Arabidopsis lectin kinase receptor gene, lecRK-al, is induced during senescence, wounding and in response to oligogalacturonic acids. Plant Physiol. Biochem. 40, 431-438.
Rudiger, H., and Gabius. H.-J. (2001). Plant lectins: Occurrence, biochemistry, functions and applications. Glycoconjugate J. 18, 589-613.
Sharon, N., and Lis, H. (2002). How protein bind carbohydrates: lessons from the legume lectins. J. Agric. Food Chem. 50, 6586-6591.
Shin, S. H., and Bleecker, A. B. (2001). Receptor-like kinases from Arabidopsis form a monophyletic gene family related to animal receptor kinases. Proc. Natl. Acad. Sci. USA 97, 10763-10768.
Shin, S. H., Karlowski, W. M., Pan, R., Tzeng, Y.-H., Mayer, K. F. X., and Li, W.-H. (2004). Comparative analysis of the receptor-like kinase family in Arabidopsis and rice. Plant Cell 16, 1220-1234.
Staswick, P. E., Su, W., and Howell, S. H. (1992). Methyl jasmonate inhibition of root growth and induction of a leaf protein are decreased in an Arabidopsis thaliana mutant. Proc. Natl. Acad. Sci. USA 89, 6837-6840.
Thompson, J. D., Higgins, D. G., and Gibson, T. J. (1994). CLUSTAL W: improving the sensitivity of progressive multiple sequence alignment through sequence weighting, position-specific gap penalties and weight matrix choice. Nucleic Acids Res. 22, 4673-4680.
Thorpe, M. R., MacRae, E. A., Minchin, P. E. H., and Edwards, C. M. (1999). Gallactose stimulation of carbon import into roots is confined to the Poaceae. J. Exper. Bot. 339, 1613-1618.
Van Damme, E. J. M., Peumans, W. J., Barre, A., and Rouger P. (1998). Plant lectins: a composite of several distinct families of structurally and evolutionary related proteins with diverse biological roles. Crit. Rev. Plant Sci. 17, 575-692.
Vijayan, M., and Chandra, N. (1999). Lectins. Curr. Opin. Struct. Biol., 9, 707-714. Ueda, J., Miyamoto, K., and Kamisaka, S. (1995). Inhibition of the synthesis of cell wall polysaccharides in oat coleoptile segments by jasmonic acid: relevance to its growth inhibition. J. Plant Growth Regul. 14, 69-76.
Yamamoto, R., Inouhe, M., and Masuda, Y. (1988). Galactose inhibition of auxin-induced growth of mono- and dicotyledonous plants. Plant Physiol. 86, 1223-1227.

What is claimed is:

1. An isolated nucleic acid selected from the group consisting of:
(a) a nucleic acid comprising the nucleotide sequence set forth in SEQ ID NO: 1;
(b) a nucleic acid comprising a nucleotide sequence that encodes the amino acid sequence of SEQ ID NO: 2; and
(c) a nucleic acid comprising an antisense nucleotide sequence corresponding to a nucleotide sequence of (a) or (b).

2. The isolated nucleic acid of claim 1, wherein the nucleic acid is DNA.

3. The isolated nucleic acid of claim 2, wherein the nucleic acid is a cDNA.

4. The isolated nucleic acid of claim 2, wherein the nucleic acid is a genomic DNA.

5. The isolated nucleic acid of claim 1, wherein the nucleic acid is RNA.

6. The isolated nucleic acid of claim 5, wherein the nucleic acid is mRNA.

7. The isolated nucleic acid of claim 1, wherein the nucleic acid is a fusion gene.

8. A vector comprising the nucleic acid of claim 1 operably linked to a promoter that controls expression in a plant cell.

9. A method for negative regulation of development in a plant, said method comprising:
transforming a plant cell with at least one nucleotide sequence operably linked to a promoter that controls expression in a plant cell, wherein the nucleotide sequence is selected from the group consisting of:
(a) a nucleic acid comprising the nucleotide sequence set forth in SEQ ID NO: 1; and
(b) a nucleic acid comprising a nucleotide sequence that encodes the amino acid sequence of SEQ ID NO: 2; and
cultivating the cell into a plant.

10. The method of claim 9, wherein the plant is a monocot.

11. The method of claim 9, wherein the development is root expansion.

12. The method of claim 11, wherein said regulation comprises transmitting signals from a sugar or a phytohormone.

13. A method for promoting increased root growth in a plant, said method comprising:
transforming a plant cell with at least one nucleotide sequence operably linked to a promoter that controls expression in a plant cell, wherein the nucleotide sequence is selected from the group consisting of:
(a) an antisense nucleotide sequence corresponding to the nucleotide sequence set forth in SEQ ID NO: 1; and
(b) an antisense nucleotide sequence corresponding to a nucleotide sequence that encodes the amino acid sequence of SEQ ID NO: 2; and
cultivating the cell into a plant.

14. The method of claim 13, wherein the plant is a monocot.

15. The method of claim 13, wherein the increased root growth comprises an expanded root system.

16. The method of claim 15, wherein the expanded root system includes more adventitious and/or longer lateral roots.

17. A transformed plant cell having stably incorporated into its genome at least one nucleotide sequence operably linked to a promoter that controls expression in a plant cell, wherein the nucleotide sequence is selected from the group consisting of:
(a) a nucleic acid comprising the nucleotide sequence set forth in SEQ ID NO: 1;
(b) a nucleic acid comprising a nucleotide sequence that encodes the amino acid sequence of SEQ ID NO: 2; and
(c) a nucleic acid comprising an antisense nucleotide sequence corresponding to a nucleotide sequence of (a) or (b).

18. A transgenic plant having stably incorporated into its genome at least one nucleotide sequence operably linked to a promoter that controls expression in a plant cell, wherein the nucleotide sequence is selected from the group consisting of:
(a) a nucleic acid having the nucleotide sequence set forth in SEQ ID NO: 1;
(b) a nucleic acid having a nucleotide sequence that encodes the amino acid sequence of SEQ ID NO: 2; and
(c) a nucleic acid having an antisense nucleotide sequence corresponding to a nucleotide sequence of (a) or (b).

19. The plant of claim 18, wherein the plant is a monocot.

20. A seed of the plant of claim 19.

21. A method for regulating development in a plant, comprising:
transforming a plant cell with at least one nucleotide sequence operably linked to a promoter that controls expression in a plant cell, wherein the nucleotide sequence is selected from the group consisting of:
(a) a nucleic acid comprising the nucleotide sequence set forth in SEQ ID NO: 1;
(b) a nucleic acid comprising a nucleotide sequence that encodes the amino acid sequence of SEQ ID NO: 2; and
(c) a nucleic acid comprising an antisense nucleotide sequence corresponding to a nucleotide sequence of (a) or (b); and
cultivating the cell into a plant.

22. The method of claim 21, wherein the plant is a monocot.

23. The method of claim 21, wherein the development comprises root growth.

24. The method of claim 21, wherein the regulating comprises introducing a modification in an aerial part of the plant.

25. A nucleotide sequence having greater than 50% homology to a full-length nucleotide sequence set forth in SEQ ID NO: 1, wherein said homologous nucleotide sequence encodes a polypeptide which retains biological activity of the full length sequence.

26. A method for negative regulation of root expansion in a plant, said method comprising:
transforming a plant cell with at least one nucleotide sequence operably linked to a promoter that controls expression in a plant cell, wherein the nucleotide sequence is selected from the group consisting of:
(a) a nucleotide sequence having greater than 50% homology to a full-length nucleotide sequence set forth in SEQ ID NO: 1, wherein said homologous nucleotide sequence encodes a polypeptide which retains biological activity of the full length sequence; and
(b) a nucleotide sequence having greater than 50% homology to a nucleotide sequence that encodes the amino acid sequence of SEQ ID NO: 2, wherein said nucleotide sequence encodes a polypeptide which retains biological activity of the full length sequence; and
cultivating the cell into a plant.

27. A method of promoting root expansion in a plant, said method comprising:
transforming a plant cell with at least one nucleotide sequence operably linked to a promoter that controls expression in a plant cell, wherein the nucleotide sequence is selected from the group consisting of:
(a) an antisense nucleotide sequence corresponding to a nucleotide sequence having greater than 50% homology to the nucleotide sequence set forth in SEQ ID NO: 1; and
(b) an antisense nucleotide sequence corresponding to a nucleotide sequence having greater than 50% homology to a nucleotide sequence that encodes the amino acid sequence of SEQ ID NO: 2; and
cultivating the cell into a plant.

28. A transformed plant cell having stably incorporated into its genome at least one nucleotide sequence operably linked to a promoter that controls expression in a plant cell, wherein the nucleotide sequence is selected from the group consisting of:
(a) a nucleotide sequence having greater than 50% homology to a full-length nucleotide sequence set forth in SEQ ID NO: 1, wherein said homologous nucleotide sequence encodes a polypeptide which retains biological activity of the full length sequence;
(b) a nucleotide sequence having greater than 50% homology to a nucleotide sequence that encodes the amino acid sequence of SEQ ID NO: 2, wherein said nucleotide sequence encodes a polypeptide which retains biological activity of the full length sequence; and
(c) an antisense nucleotide sequence corresponding to a nucleotide sequence of (a) or (b).

29. A transgenic plant having stably incorporated into its genome at least one nucleotide sequence operably linked to a promoter that controls expression in a plant cell, wherein the nucleotide sequence is selected from the group consisting of:
(a) a nucleotide sequence having greater than 50% homology to a full-length nucleotide sequence set forth in SEQ ID NO: 1, wherein said homologous nucleotide sequence encodes a polypeptide which retains biological activity of the full length sequence;
(b) a nucleotide sequence having greater than 50% homology to a nucleotide sequence that encodes the amino acid sequence of SEQ ID NO: 2, wherein said nucleotide sequence encodes a polypeptide which retains biological activity of the full length sequence; and
(c) an antisense nucleotide sequence corresponding to a nucleotide sequence of (a) or (b).

30. A seed of the plant of claim 29.

31. A transgenic plant whose genome comprises a disruption of the Oryza sativa Lectin-Like Receptor Kinase 1 (Oslrk1) gene, wherein the disruption comprises a Ds insertion, and wherein the disruption results in said transgenic plant exhibiting increased root growth as compared to a wild-type plant.

32. The transgenic plant of claim 31, wherein the disruption is a complete knockout of the Oslrk1 gene.

33. The transgenic plant of claim 31, wherein the increased root growth comprises an expanded root system.

34. The transgenic plant of claim 33, wherein the expanded root system includes more adventitious or longer lateral roots.

35. The transgenic plant of claim 31, wherein the transgenic plant exhibits modifications in its aerial part said modifications selected from the group consisting of larger leaves, delayed flowering and higher seed yield as compared to a wild-type plant.

36. The transgenic plant of claim 35, wherein the higher seed yield is a yield that is about 21% higher than that of a wild-type plant.

37. The transgenic plant of claim 31, wherein the transgenic plant exhibits a hypersensitivity to D-galactose as compared to that of a wild-type plant.

38. The transgenic plant of claim 31, wherein the Ds insertion is located 117 bp downstream of the ATG codon in the first exon of the Oslrk1 gene.

39. The transgenic plant of claim 35, wherein the flowering is delayed by about five to about seven days as compared to the flowering in a wild-type plant.

40. The transgenic plant of claim 31, wherein the transgenic plant, when mature, is about 30% taller than a wild-type plant grown in similar conditions.

41. The transgenic plant of claim 31, wherein the transgenic plant, when mature, exhibits about 70% more branches in the panicles than a wild-type plant grown in similar conditions.

42. The transgenic plant of claim 31, wherein the transgenic plant, at the seedling stage, exhibits a shoot length of about two times that of a wild-type plant grown in similar conditions.

43. The transgenic plant of claim 31, wherein said plant, at the seedling stage, exhibits about 56% more adventitious lateral roots than a wild-type plant grown in similar conditions.

44. The transgenic plant of claim 31, wherein said plant, at the seedling stage, exhibits lateral roots that are about 74% longer than lateral roots of a wild-type plant grown in similar conditions.

45. An isolated nucleic acid comprising an Oryza sativa Lectin-Like Receptor Kinase 1 (Oslrk1) gene, wherein said gene comprises a Ds insertion located about 117 bp downstream of the ATG codon in its first exon.

46. A seed of the plant of claim 31.

47. A method for increasing root growth in a plant, said method comprising:
manipulating the genome of a plant cell to comprise a disruption of the Oryza sativa Lectin-Like Receptor Kinase 1 (Oslrk1) gene, wherein the disruption comprises a Ds insertion; and
cultivating the cell into a plant.

48. The method of claim 47, wherein the disruption is a complete knockout of the (Oslrk1) gene.

49. The method of claim 47, wherein the increased root growth comprises an expanded root system.

50. The method of claim 49, wherein the expanded root system includes more adventitious or longer lateral roots.

51. The method of claim 47, wherein the Ds insertion is located 117 bp downstream of the ATG codon in the first exon of the Oslrk1 gene.

52. A method of modifying the aerial part of a plant, said method comprising:
manipulating the genome of a plant cell to comprise a disruption of the Oryza sativa Lectin-Like Receptor Kinase 1 (Oslrk1) gene, wherein the disruption comprises a Ds insertion; and
cultivating the cell into a plant.

53. The method of claim 52, wherein the disruption is a complete knockout of the Oslrk1 gene.

54. The method of claim 52, wherein the modifications in the aerial part of the plant are selected from the group consisting of larger leaves, delayed flowering and higher seed yield as compared to a wild-type plant.

55. The method of claim 54, wherein the higher seed yield is a yield that is about 21% higher than that of a wild-type plant.

56. A method of increasing sensitivity to D-galactose in a plant, said method comprising:
manipulating the genome of a plant cell to comprise a disruption of the Oryza sativa Lectin-Like Receptor Kinase 1 (Oslrk1) gene, wherein the disruption comprises a Ds insertion; and
cultivating the cell into a plant.

57. The method of claim 56, wherein the Ds insertion is located 117 bp downstream of the ATG codon in the first exon of the Oslrk1 gene.

58. The method of claim 56, wherein the disruption is a complete knockout of the Oslrk1 gene.

59. The transgenic plant of claim 31, wherein the plant is a monocot.

60. An isolated Ds insertion mutant of the Oryza sativa Lectin-Like Receptor Kinase 1 (Oslrk1) gene.

61. The Ds insertion mutant of claim 60, wherein the Ds insertion is located 117 bp downstream of the ATG codon in the first exon of the Oslrk1 gene.

62. A method for increasing root growth in a plant, said method comprising:
manipulating the genome of a plant cell to comprise a disruption of the Oryza sativa Lectin-Like Receptor Kinase 1 (Oslrk1) gene, wherein the disruption comprises an inactivation of the Oslrk1 gene; and
cultivating the cell into a plant.

63. The method of claim 62, wherein inactivation is by degradation of the gene's transcript.

64. The method of claim 63, wherein the degradation is achieved via an miRNA, RNAi, or sRNA technique.
